(19) United States
(12) Patent Application Publication
(10) Pub. No.: US 2026/0060193 A1
(43) Pub. Date: Mar. 5, 2026

(54) HYDROGEL-CARRIERS FOR INDUCTION OF PLANT MORPHOGENESIS

(71) Applicant: KEYGENE N.V., Wageningen (NL)

(72) Inventors: Hugo Ferdinand HOFHUIS, Wageningen (NL); Raymond Jozef Maurinus HULZINK, Wageningen (NL)

(73) Assignee: KEYGENE N.V., Wageningen (NL)

(21) Appl. No.: 19/106,659

(22) PCT Filed: Sep. 1, 2023
(86) PCT No.: PCT/EP2023/073987

§ 371 Date: Feb. 26, 2025

(30) Foreign Application Priority Data
Sep. 1, 2022 (EP) 22193482.1
Dec. 8, 2022 (EP) 22212182.4

(51) Int. Cl. A01H 4/00; C12N 15/82

(52) U.S. Cl. CPC A01H 4/002; A01H 4/008; C12N 15/8205; C12N 15/8238; C12N 2820/002

(57) ABSTRACT

The invention pertains to a method for producing a plant shoot, wherein said method comprises the step of locally contacting a plant tissue comprising a sequence encoding at least one regeneration factor under the control of an inducible promoter with a inductive hydrogel. The invention further pertains to an inductive hydrogel and the use of said inductive hydrogel for inducing regeneration.

### FIELD OF THE INVENTION

The invention relates to the field of molecular plant biology, in particular in the field of plant regeneration. The invention concerns methods for improving regeneration of plant tissue by locally applying inducers of morphogenic regulators.

### BACKGROUND

Regeneration, the process of growing a whole plant from a single or a group of cells, is often a bottleneck in plant biotechnological workflows. Non-limiting examples of such biotechnological workflows are the general multiplication of (clonally propagated, haploid, polyploidy and/or heterozygous F1) plant material, but also more advanced plant biotechnology workflows such as, but not limited to, targeted plant genome editing, production of (stable or transient) transformants and doubled haploid induction.

Inducible constructs comprising regeneration factors have been applied relatively effectively in different plant types. These (trans-) genes, also indicated as morphogenic genes, have been shown to induce shoot regeneration and/or somatic embryogenesis in plant tissue and/or explants (see e.g., WO2019/211296 and WO2017/074547, which are incorporated herein by reference). Non-limiting examples of such genes are genes encoding WUS/WOX homeobox polypeptides (van der Graaff et al., 2009, Genome Biology 10:248), transcription factors comprising two AP2 DNA binding domains, PLETHORA (PLT) proteins and related polypeptides like Ovule Development Protein 2 (ODP2) and Babyboom (BBM), WOUND INDUCED DEDIFFERENTIATION 1 (WIND1) protein, SHORT ROOT (SHR) protein, and SCARECROW (SCR) protein (Galinha et al., 2007 Nature 449:1053-1057; Wildwater et al., 2005, Cell 123, 1337-1349).

In many instances, expression of such (trans-)genes in explants, for instance leaves, results in the appearance of incipient somatic embryos, however, such embryos often do not develop into whole plants. Therefore there is a need for an improved protocol for inducing regeneration in plant explants that result in the formation of a mature plant.

### SUMMARY

Embodiment 1. A method for producing a plant shoot, comprising the steps of:
a) providing a plant tissue comprising a sequence encoding at least one regeneration factor under the control of an inducible promoter;
b) locally contacting the plant tissue to an inductive hydrogel bead comprising an inducer, wherein the inducer is a compound capable of inducing the inducible promoter, optionally upon binding to a trans-activator comprised in the plant tissue; and
c) allowing the shoot to be produced from the plant tissue.

Embodiment 2. A method for producing a plant shoot according to embodiment 1, wherein the bead is a macrosphere having a diameter of about 0.1-10 millimetre (mm), or a microsphere having a diameter of about 0.1-100 micrometre (μm).

Embodiment 3. A method for producing a plant shoot according to embodiment 1 or 2, wherein the inducer is a steroid,

Embodiment 4. A method for producing a plant shoot according to embodiment 3, wherein the steroid is at least one of dexamethasone or a derivative thereof and wherein the steroid is β-estradiol or a derivative thereof.

Embodiment 5. A method according to any one of the preceding embodiments,
wherein the inducer is dexamethasone or a derivative thereof, and wherein the plant tissue of step a) comprises trans-activator that is a GVG protein; and/or,
wherein the inducer is β-estradiol or a derivative thereof, and wherein the plant tissue of step a) comprises trans-activator that is a XVE protein.

Embodiment 6. A method according to any one of the preceding embodiments, wherein the concentration of the inducer in the hydrogel bead is about 1 nM-100 μM.

Embodiment 7. A method according to any one of the preceding embodiments, wherein the at least one regeneration factor comprises at least one of a PLETHORA (PLT) polypeptide and a WUS/WOX homeobox polypeptide, wherein preferably:
i) the PLT polypeptide is selected from the group consisting of PLT1, PLT2, PLT3, PLT4, PLT5 and PLT7, preferably PLT1; and
ii) the WUS/WOX homeobox polypeptide is selected from the group consisting of WUS1, WUS2, WUS3, WOX2A, WOX4, WOX5, or WOX9, preferably WOX5.

Embodiment 8. A method according to embodiment 7, wherein the at least one regeneration factor comprises PLT1 and WOX5, wherein the PLT1- and WOX5-encoding sequences are each operably linked to an inducible promoter, preferably a GVG-inducible promoter.

Embodiment 9. A method according to any one of the preceding embodiments, wherein the at least one regeneration factor comprises WIND1, and wherein the WIND1-encoding sequence is operably linked to an inducible promoter, preferably XVE-inducible promoter.

Embodiment 10. A method according to any one of preceding embodiments, wherein the hydrogel is an alginate or a derivative thereof.

Embodiment 11. A method according to any one of the preceding embodiments, wherein the plant tissue in step a) comprises an expression construct encoding the at least one regeneration factor under the control of an inducible promoter, and optionally encoding a trans-activator.

Embodiment 12. A method according to any one of the preceding embodiments, wherein the plant tissue of step a) is a hypocotyl or a leaf, wherein the pant tissue is preferably any one of a cotyledon, a first true leaf, and a young (axillary) leaf.

Embodiment 13. A method according to any one of the preceding embodiments, further comprising a step of regenerating a plant from the produced plant shoot.

Embodiment 14. A combination of a hydrogel bead comprising an inducer of gene expression as defined in any one of embodiments 1-9 and a plant cell.

Embodiment 15. Use of a hydrogel bead according to embodiment 14 for inducing regeneration of a plant tissue.

### LEGENDS TO THE FIGURES

FIG. 1 Hydrogel-carriers for induction of regeneration in S. lycopersicum (A) alginate macrospheres. (B) inductive alginate bead positioned on the surface of a young leaf explant. (C) Local morphogenic responses two and a half week after inductive bead application. Boxed region corresponds to the boxed region depicted in (B). (D) Example of the typical morphogenic response observed in conventional induction assays. (E) Formation of embryonic structures (indicated by *) after local hydrogel bead application. (F) Resulting regenerant shoots.

FIG. 2 Hydrogel-carriers for induction of regeneration in C. Annuum c.v. Maor. (A) inductive alginate bead positioned on a leaf-like surface. (B) Local morphogenic responses 19 days after inductive bead application. (C-G) Developmental stages in Maor somatic embryo development. (C) Inception-(D) Globular-(E) heart-(F) Early torpedo- and (G) Late torpedo stage. (H) Regenerating shoot from embryonic structures while attached to callus. (I) Resulting regenerant shoot. Note the presence of a root system and internodes. (J) Fluorescent recording of the shoot apex of the regenerant depicted in (I). Note the presence of a regular phylotactic pattern. (K) Maor regenerant with mature transgenic fruits. (L-N) Subsequent stages of flower development during fruit set. (L) Closed flower bud. (M) Open flower bud. (N) Fertilized flower bud. (O) Mature transgenic fruit with transgenic seed set. (P) Transgenic Maor seed. Green fluorescent signal reports presence of the Stem Cell Niche transgene.

### DEFINITIONS

Various terms relating to the methods, compositions, uses and other aspects provided in the specification and claims are to be given their ordinary meaning in the art, unless otherwise indicated. Other specifically defined terms are to be construed in a manner consistent with the definition provided herein.

Methods of carrying out the conventional techniques used in methods provided herein will be evident to the skilled worker. The practice of conventional techniques in molecular biology, biochemistry, computational chemistry, cell culture, recombinant DNA, bioinformatics, genomics, sequencing, breeding by crossing and selection, and related fields are well-known to those of skill in the art and are discussed, for example, in the following literature references: Sambrook et al. Molecular Cloning. A Laboratory Manual, 2nd Edition, Cold Spring Harbor Laboratory Press, Cold Spring Harbor, N. Y., 1989; Ausubel et al., Current Protocols in Molecular Biology, John Wiley & Sons, New York, 1987 and periodic updates; and the series Methods in Enzymology, Academic Press, San Diego.

The singular terms “a,” “an,” and “the” include plural referents unless the context clearly dictates otherwise. Thus, for example, reference to “a cell” includes a combination of two or more cells, and the like. The indefinite article “a” or “an” thus usually means “at least one”.

The term “and/or” refers to a situation wherein one or more of the stated cases may occur, alone or in combination with at least one of the stated cases, up to with all of the stated cases.

As used herein, the term “about” is used to describe and account for small variations. For example, the term can refer to less than or equal to ±(+ or −) 10%, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

The term “comprising” is construed as being inclusive and open ended, and not exclusive. Specifically, the term and variations thereof mean the specified features, steps or components are included. These terms are not to be interpreted to exclude the presence of other features, steps or components.

The terms “protein” or “polypeptide” are used interchangeably and refer to molecules consisting of a chain of amino acids, without reference to a specific mode of action, size, 3 dimensional structure or origin. A “fragment” or “portion” of a protein may thus still be referred to as a “protein”. An “isolated protein” is used to refer to a protein which is no longer in its natural environment, for example in vitro or in a recombinant bacterial or plant host cell.

“Plant” refers to either the whole plant or to parts of a plant tissue or organs (e.g. pollen, seeds, roots, stems, leaves, flowers, flower buds, anthers, fruit, etc.) obtainable from the plant, as well as derivatives of any of these and progeny derived from such a plant by selfing or crossing or apomictic reproduction. Non-limiting examples of plants include crop plants and cultivated plants, such as African eggplant, alliums, artichoke, asparagus, barley, beet, bell pepper, bitter gourd, bladder cherry, bottle gourd, cabbage, cannabis, canola, carrot, cassava, cauliflower, celery, chicory, common bean, corn salad, cotton, cucumber, eggplant, endive, fennel, gherkin, grape, hop, hot pepper, lettuce, maize, melon, oilseed rape, okra, parsley, parsnip, pepino, pepper, potato, pumpkin, radish, rice, ridge gourd, rocket, rye, snake gourd, sorghum, spinach, sponge gourd, squash, sugar beet, sugar cane, sunflower, tomatillo, tomato, tomato rootstock, vegetable Brassica, watermelon, wax gourd, wheat and zucchini.

“Plant cell(s)” include protoplasts, gametes, suspension cultures, microspores, pollen grains, etc., either in isolation or within a tissue, organ or organism, from plant origin. The plant cell can e.g. be part of a multicellular structure, such as a callus, meristem, plant organ or an explant. A plant cell may be a meristematic cell, a somatic cell and/or a reproductive cell.

“Similar conditions” for culturing the plant/plant cells means among other things the use of a similar temperature, humidity, nutrition and light conditions, and similar irrigation and day/night rhythm.

The terms “homology”, “sequence identity” and the like are used interchangeably herein. Sequence identity is herein defined as a relationship between two or more amino acid (polypeptide or protein) sequences or two or more nucleotide (polynucleotide) sequences, as determined by comparing the sequences. In the art, “identity” also means the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods. The percentage sequence identity/similarity can be determined over the full length of the sequence.

As used herein “sequence identity” refers to the extent to which two optimally aligned polynucleotide or peptide sequences are invariant throughout a window of alignment of components, e.g., nucleotides or amino acids. An “identity fraction” for aligned segments of a test sequence and a reference sequence is the number of identical components which are shared by the two aligned sequences divided by the total number of components in reference sequence segment, i.e., the entire reference sequence or a smaller defined part of the reference sequence. “Percent identity” is the identity fraction times 100.

“Sequence identity” and “sequence similarity” can be determined by alignment of two peptide or two nucleotide sequences using global or local alignment algorithms, depending on the length of the two sequences. Sequences of similar lengths are preferably aligned using a global alignment algorithms (e.g. Needleman Wunsch) which aligns the sequences optimally over the entire length, while sequences of substantially different lengths are preferably aligned using a local alignment algorithm (e.g. Smith Waterman). Sequences may then be referred to as “substantially identical” or “essentially similar” when they (when optimally aligned by for example the programs GAP or BESTFIT using default parameters) share at least a certain minimal percentage of sequence identity (as defined herein). The percent of sequence identity is preferably determined using the “BESTFIT” or “GAP” program of the Sequence Analysis Software Package™ (Version 10; Genetics Computer Group, Inc., Madison, Wis.). GAP uses the Needleman and Wunsch global alignment algorithm (Needleman and Wunsch, Journal of Molecular Biology 48:443-453, 1970) to align two sequences over their entire length (full length), maximizing the number of matches and minimizing the number of gaps. A global alignment is suitably used to determine sequence identity when the two sequences have similar lengths. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, CA 92121-3752 USA, or using open source software, such as the program “needle” (using the global Needleman Wunsch algorithm) or “water” (using the local Smith Waterman algorithm) in EmbossWIN version 2.10.0, using the same parameters as for GAP above, or using the default settings (both for ‘needle’ and for ‘water’ and both for protein and for DNA alignments, the default Gap opening penalty is 10.0 and the default gap extension penalty is 0.5; default scoring matrices are Blossum62 for proteins and DNAFull for DNA). gap extension penalty is 0.5; default scoring matrices are Blossum62 for proteins and DNAFull for DNA). “BESTFIT” performs an optimal alignment of the best segment of similarity between two sequences and inserts gaps to maximize the number of matches using the local homology algorithm of Smith and Waterman (Smith and Waterman, Advances in Applied Mathematics, 2:482-489, 1981, Smith et al., Nucleic Acids Research 11:2205-2220, 1983). When sequences have a substantially different overall lengths, local alignments, such as those using the Smith Waterman algorithm, are preferred.

Useful methods for determining sequence identity are also disclosed in Guide to Huge Computers, Martin J. Bishop, ed., Academic Press, San Diego, 1994, and Carillo, H., and Lipton, D., Applied Math (1988) 48:1073. More particularly, preferred computer programs for determining sequence identity include the Basic Local Alignment Search Tool (BLAST) programs which are publicly available from National Center Biotechnology Information (NCBI) at the National Library of Medicine, National Institute of Health, Bethesda, Md. 20894; see BLAST Manual, Altschul et al., NCBI, NLM, NIH; Altschul et al., J. Mol. Biol. 215:403-410 (1990); version 2.0 or higher of BLAST programs allows the introduction of gaps (deletions and insertions) into alignments; for peptide sequence BLASTX can be used to determine sequence identity; and, for polynucleotide sequence BLASTN can be used to determine sequence identity.

Alternatively percentage similarity or identity may be determined by searching against public databases, using algorithms such as FASTA, BLAST, etc. Thus, the nucleic acid and protein sequences described herein can further be used as a “query sequence” to perform a search against public databases to, for example, identify other family members or related sequences. Such searches can be performed using the BLASTn and BLASTx programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to nucleic acid molecules described herein. BLAST protein searches can be performed with the BLASTx program, score=50, wordlength=3 to obtain amino acid sequences homologous to protein molecules described herein. To obtain gapped alignments for comparison purposes, Gapped BLAST can be utilized as described in Altschul et al., (1997) Nucleic Acids Res. 25 (17): 3389-3402. When utilizing BLAST and Gapped BLAST programs, the default parameters of the respective programs (e.g., BLASTx and BLASTn) can be used. See the homepage of the National Center for Biotechnology Information at http://www.ncbi.nlm.nih.gov/.

A “nucleic acid” or “polynucleotide” as used herein may include any polymer or oligomer of pyrimidine and purine bases, preferably cytosine, thymine, and uracil, and adenine and guanine, respectively (See Albert L. Lehninger, Principles of Biochemistry, at 793-800 (Worth Pub. 1982) which is herein incorporated by reference in its entirety for all purposes). Contemplated are any deoxyribonucleotide, ribonucleotide or nucleic acid component, and any chemical variants thereof, such as methylated, hydroxymethylated or glycosylated forms of these bases, and the like. The polymers or oligomers may be heterogeneous or homogenous in composition, and may be isolated from naturally occurring sources or may be artificially or synthetically produced. In addition, the nucleic acids may be DNA (optionally cDNA) or RNA, or a mixture thereof, and may exist permanently or transitionally in single-stranded or double-stranded form, including homoduplex, heteroduplex, and hybrid states.

An “isolated nucleic acid” is used to refer to a nucleic acid which is no longer in its natural environment, for example in vitro or in a recombinant bacterial or plant cell. A nucleic acid and/or protein may be at least one of a recombinant, synthetic or artificial nucleic acid and/or protein.

The term “expression construct” (or nucleic acid construct or vector) is used to refer to as a man-made nucleic acid molecule resulting from the use of recombinant DNA technology. The terms “nucleic acid construct”, “nucleic acid vector” or “expression construct” therefore do not include naturally occurring nucleic acid molecules although a nucleic acid construct may comprise (parts of) naturally occurring nucleic acid molecules.

The vector backbone may for example be a binary or superbinary vector (see e.g. U.S. Pat. No. 5,591,616, US2002138879 and WO 95/06722), a co-integrate vector or a T-DNA vector, as known in the art and as described elsewhere herein, into which a chimeric gene is integrated or, if a suitable transcription regulatory sequence is already present, only a desired nucleic acid sequence (e.g. a coding sequence, an antisense or an inverted repeat sequence) is integrated downstream of the transcription regulatory sequence. Vectors can comprise further genetic elements to facilitate their use in molecular cloning, such as e.g. selectable markers, multiple cloning sites and the like.

The term “gene” means a DNA fragment comprising a region (transcribed region), which is transcribed into an RNA molecule (e.g. an mRNA) in a cell, operably linked to suitable regulatory regions (e.g. a promoter). A gene will usually comprise several operably linked fragments, such as a promoter, a 5′ leader sequence, a coding region and a 3′ non-translated sequence (3′ end) comprising a polyadenylation site.

“Expression of a gene” refers to the process wherein a DNA region which is operably linked to appropriate regulatory regions, particularly a promoter, is transcribed into an RNA, which is biologically active, e.g. which is capable of being translated into a biologically active protein or peptide, or e.g. a regulatory non-coding RNA.

The term “operably linked” refers to a linkage of polynucleotide elements in a functional relationship. A nucleic acid is “operably linked” when it is placed into a functional relationship with another nucleic acid sequence. For instance, a promoter, or rather a transcription regulatory sequence, is operably linked to a coding sequence if it affects the transcription of the coding sequence. Operably linked may mean that the DNA sequences being linked are contiguous.

“Promoter” refers to a nucleic acid fragment that functions to control the transcription of one or more nucleic acids. A promoter fragment is preferably located upstream (5′) with respect to the direction of transcription of the transcription initiation site of the gene, and is structurally identified by the presence of a binding site for DNA-dependent RNA polymerase, transcription initiation site(s) and can further comprise any other DNA sequences, including, but not limited to transcription factor binding sites, repressor and activator protein binding sites, and any other sequences of nucleotides known to one of skill in the art to act directly or indirectly to regulate the amount of transcription from the promoter.

A “constitutive” promoter is a promoter that is active in most tissues under most physiological and developmental conditions. An “inducible” promoter is a promoter that is physiologically (e.g. by external application of certain compounds or “inducers”) or developmentally regulated. A “tissue specific” promoter is only active in specific types of tissues or cells.

Optionally the term “promoter” may also include the 5′ UTR region (5′ Untranslated Region) (e.g. the promoter may herein include one or more parts upstream of the translation initiation codon of transcribed region, as this region may have a role in regulating transcription and/or translation).

A “3′ UTR” or “3′ non-translated sequence” (also often referred to as 3′ untranslated region, or 3′end) refers to the nucleic acid sequence found downstream of the coding sequence of a gene, which comprises for example a transcription termination site and (in most, but not all eukaryotic mRNAs) a polyadenylation signal (such as e.g. AAUAAA or variants thereof). After termination of transcription, the mRNA transcript may be cleaved downstream of the polyadenylation signal and a poly(A) tail may be added, which is involved in the transport of the mRNA to the cytoplasm (where translation takes place).

The term “cDNA” means complementary DNA. Complementary DNA is made by reverse transcribing RNA into a complementary DNA sequence. cDNA sequences thus correspond to RNA sequences that are expressed from genes. As RNA sequences expressed from the genome can undergo splicing, i.e. introns are spliced out of the pre-mRNA and exons are joined together, before being translated in the cytoplasm into proteins, it is understood that expression of a cDNA means expression of the mRNA that encodes for the cDNA. The cDNA sequence thus may not be identical to the genomic DNA sequence to which it corresponds as the cDNA may encode only the complete open reading frame, consisting of the joined exons, for a protein, whereas the genomic DNA sequence may comprise exon sequences interspersed by intron sequences. Genetically modifying a gene which encodes a protein may thus not only relate to modifying the sequences encoding the protein, but may also involve mutating intronic sequences of the genomic DNA and/or other gene regulatory sequences of that gene.

The term “regeneration” is herein defined as the formation of a new tissue and/or a new organ from a single plant cell, a group of cells, a callus, an explant, a tissue or from an organ. Regeneration may include the formation of a new plant from a single plant cell or from e.g. a callus, an explant, a tissue or an organ. The plant cell for regeneration can be an undifferentiated plant cell. A preferred plant cell is a protoplast. The regeneration process can occur directly from parental tissues or indirectly, e.g. via the formation of a callus. The regeneration pathway can be somatic embryogenesis or organogenesis. Somatic embryogenesis is understood herein as the formation of somatic embryos, which can be grown into whole plants. Organogenesis is understood herein as the formation of new organs from (undifferentiated) cells. Organogenesis may be at least one of meristem formation, adventitious shoot formation, inflorescence formation, root formation, elongation of adventitious shoots and (subsequent) the formation of a complete plant. Preferably, regeneration is at least one of shoot regeneration, (ectopic) apical meristem formation and root regeneration. Shoot regeneration as defined herein is de novo shoot formation. For example, regeneration can be the regeneration of a(n) (inflorescence) shoot from a(n) (elongated) hypocotyl explant.

The term “normal growth conditions” is herein understood as an environment wherein a plant grows. Such conditions include at minimum a suitable temperature (i.e. between 0° C.-60° C.), nutrition, day/night rhythm and irrigation.

The term “conditions that allow for regeneration” is herein understood as an environment wherein a plant cell or tissue can regenerate, preferably including normal growth conditions.

“Shoot organogenesis” is the regeneration pathway by which cells, preferably cells of callus or explant, form a de novo shoot apical meristem that develops into a shoot with leaf primordia and leaves. As there is only one apical meristem, this is a unipolar structure, and roots are not formed at this stage. The vascular system of the shoot is often connected to the parent tissue. Only after the shoots have fully formed and elongated, and are taken off e.g. the callus or explant, can the formation of roots be induced in a separate root induction step on a different culture medium (Thorpe, T A (1993) In vitro Organogenesis and Somatic Embryogenesis: Physiological and Biochemical Aspects. In: Roubelakis-Angelakis K. A., Van Thanh K. T. (eds) Morphogenesis in Plants. NATO ASI Series (Series A: Life Sciences), Vol. 253. Springer, Boston, MA).

Shoot organogenesis may occur spontaneously, i.e. without the external addition of any plant growth regulators (PGRs). Shoot organogenesis may be induced by plant growth regulators, usually cytokinins alone in different concentrations or in combination with an auxin, wherein preferably the cytokinins remain a constituent of the culture media until the new shoot apical meristems and the shoots have been formed and are sufficiently elongated, e.g. to take them off the primary explant or callus. Preferably, for the induction of shoot formation, the concentration of cytokinins exceeds the concentration of auxins.

“Somatic embryogenesis” leads to the formation of bipolar structures resembling zygotic embryos, which contain a root-shoot axis with a closed independent vascular system. In other words, both root and shoot primordia are being formed simultaneously, and there is no vascular connection to the underlying tissue (Dodds, J H and Roberts, L W (1985) Experiments in plant tissue culture. Cambridge University Press, Cambridge, UK). Somatic embryogenesis can e.g. be induced indirectly from callus or cell suspensions, or they can be induced directly on cells of explants (Thorpe, supra). Somatic embryo formation passes through a number of distinct stages, from globular stage (small isodiametric cell clusters), via heart stage (bilaterally symmetrical structures) to torpedo stage (elongation). The globular-to-heart transition is marked by the outgrowth of the two cotyledons and the beginning of the development of the radicle (Zimmerman, J L (1993) Somatic Embryogenesis: A Model for Early Development in Higher Plants. The Plant Cell 5:1411-1423; Von Arnold et al (2002) Developmental pathways of somatic embryogenesis. Plant Cell, Tissue and Organ Culture 69:233-249). Finally, torpedo-stage somatic embryos can develop into plantlets that contain green cotyledons, elongated hypocotyls, and developed radicles with clearly differentiated root hairs (Zimmerman, supra), in a process that is termed ‘germination’ (analogous to zygotic embryos) or ‘conversion’ or ‘maturation’ (Von Arnold et al., supra). In the induction of somatic embryogenesis, directly or indirectly, preferably auxins are used at the initial stage to induce an embryogenic state in the callus, but the embryos form after passage of the culture to a medium without or with reduced auxin levels. Auxins used for somatic embryo induction are e.g. 1-naphthaleneacetic acid (NAA), 2,4-dichlorophenoxyacetic acid (2,4-D), picloram and dicamba.

The term “endogenous” as used herein in combination with a protein or nucleic acid means that said protein or nucleic acid is still contained within the plant, i.e. is present in its natural environment. Often an endogenous gene will be present in its normal genetic context in the plant.

“Plant hormones”, “plant growth hormone”, “plant growth regulator” or “phytohormone” is a chemical that influences the growth and/or development of plant cells and tissues. Plant growth regulators comprise chemicals from the following five groups: auxins, cytokinins, gibberellins, abscisic acid (ABA) and ethylene. In addition to the five main groups, two other classes of chemical are often regarded as plant growth regulators: brassinosteroids and polyamines.

“Targeted mutagenesis” is mutagenesis that can be designed to alter a specific nucleotide or nucleic acid sequence, such as but not limited to, oligonucleotide-directed mutagenesis, mutagenesis using RNA-guided endonucleases (e.g. the CRISPR-technology), meganucleases, TALENs or Zinc finger technology.

The term “sequence of interest” includes, but is not limited to, any genetic sequence preferably present within a cell, such as, for example a gene, part of a gene, or a non-coding sequence within or adjacent to a gene. The sequence of interest may be present in a chromosome, an episome, an organellar genome such as mitochondrial or chloroplast genome or genetic material that can exist independently to the main body of genetic material such as an infecting viral genome, plasmids, episomes, transposons for example. A sequence of interest may be within the coding sequence of a gene, within transcribed non-coding sequence such as, for example, leader sequences, trailer sequence or introns. Said sequence of interest may be present in a double or a single strand nucleic acid molecule. The nucleic acid sequence is preferably present in a double-stranded nucleic acid molecule. The sequence of interest may be any sequence within a nucleic acid, e.g., a gene, gene complex, locus, pseudogene, regulatory region, highly repetitive region, polymorphic region, or portion thereof. The sequence of interest may also be a region comprising genetic or epigenetic variations indicative for a phenotype or disease. Preferably, the sequence of interest is a small or longer contiguous stretch of nucleotides (i.e. a polynucleotide) of duplex DNA, wherein said duplex DNA further comprises a sequence complementary to the sequence of interest in the complementary strand of said duplex DNA. The sequence of interest may be, or may be part of, a gene of interest, preferably an endogenous gene of interest.

### DETAILED DESCRIPTION

The inventors discovered a versatile, straightforward and improved protocol for regeneration of plant tissues, preferably explants. To this end, the inventors developed a method wherein a plant tissue comprising one or more sequences encoding a regeneration factor under the control of an inducible promoter is locally brought into contact with a hydrogel matrix comprising an inducing compound (indicated herein as an inductive hydrogel), i.e. a compound that is capable of activating or inducing (directly or indirectly) the inducible promoter operably linked to one or more sequences encoding a regeneration factor. The inventors discovered that the local application of such inductive hydrogel on an explant resulted in effective plant cell regeneration. For instance, local application of an inductive hydrogel on a plant leaf comprising a construct encoding at least one regeneration factor under control of an inducible promoter results in formation of a shoot that can be grown in a viable seedling (i.e. a whole plant). Preferably said shoot formation is under conditions free of auxins, even more preferably fee of plant hormones.

Without wishing to be bound by a theory, the hydrogel may provide for a dose gradient of the inducing compound in the plant tissue, wherein said dose can be a spatial or a temporal gradient that diminishes over space or time. Therefore provided is an easy and straightforward method to apply compounds to a plant tissue in a non-uniform dose dependent way, e.g. both spatial and temporal application. The method as defined herein preferably comprises a step of locally contacting the plant tissue to an inductive hydrogel comprising an inducer to obtain a concentration gradient of the inducer. Preferably the concentration gradient is a spatial and/or a temporal gradient.

Local application of inducing agents have been described in the art. For instance, spraying, painting and lanolin paste have been used for local application in plants (Samalova et al., The Plant Journal, 2005; 41, 919-935; Borghi, Plant Developmental Biology: Methods and Protocols”, vol. 655, pages 65-75; and Wyrzykowska et al. Plant Physiology, August 2006, Vol. 141, pp. 1338-1348), preferably in presence of a surface tension reducing agent such as Silwet L-77 (Samalova et al., supra; Borghi, supra) However, the use of surface tension reducing agents like Silwet L-77 in spraying and/or the use of sticky pasts like lanolin (requiring heating before application) are unfavourable for fragile plant material like leaf structures. Furthermore, these are laborious procedures and not straightforward for the local application of inducing agents.

Provided is a method to regenerate shoot and/or a method to regenerate plants, wherein said method comprises the step of locally applying an inductive hydrogel to a plant tissue. Preferably, said plant tissue comprises at least one regeneration factor under the control of an inducible promoter, and the inductive hydrogel comprises a compound (inducer) that can induce or activate the inducible promoter, optionally through binding of a trans-activator present in a plant tissue. Preferably at least part of the cells of said plant tissue comprises a construct encoding said at least one regeneration factor under the control of an inducible promoter and optionally a transactivator for inducing said inducible promoter upon binding of an inducer. Optionally said transactivator is encoded on a construct (optionally the same construct encoding the at least one regeneration factor and the inducible promoter operably linked thereto) comprised in said at least part of the cells of said plant tissue, which further may comprise a promoter that is constitutive active in plant cells that is operably linked to the sequence encoding the transactivator. Preferably, said construct is an isolated construct.

Optionally said construct is present (preferably stably inserted into the genome) of substantially all cells of said plant tissue. Therefore, preferably said plant tissue is from a stable transgenic line having said construct stably inserted in its genome.

Preferably, at least part of the cells of said plant tissue comprises at least one regeneration factor under the control of an inducible promoter, and the inductive hydrogel comprises a compound (inducer) that can induce (activate) the inducible promoter, optionally through binding of a trans-activator present in the cells of the plant tissue. The inductive hydrogel is preferably produced by dissolving an inducer in the hydrogel and the local application is preferably performed by applying the inductive hydrogel to only part of the surface of the plant tissue, for instance by applying the inductive hydrogel as a bead (sphere or granule) on the plant tissue. Preferably, the inductive hydrogel is only contacted to at most about 45%, 40%, 35%, 30%, 25%, 20%, 10%, or 5% of the surface of the plant tissue of the method provided herein. Preferably, the inductive hydrogel is only contacted to less than 50% of the surface of the plant tissue, preferably less than about 50%, 45%, 40%, 35%, 30%, 25%, 20%, 5%, 10%, or 5%. Preferably said plant tissue is a dissected leaf. In a preferred embodiment, the compound is an inducer of gene expression.

When the inducer was used to express factors involved in regeneration, the inventors noticed that the method provided herein can enable shoot formation and outgrowth, overcoming the problem of developmental arrest of the induced organs. Therefore, preferably, using the method of the invention shoot formation is increased as compared to a method that is otherwise similar but that differs in that in step b) at least one whole surface of the plant tissue is exposed to an inducing hydrogel, mostly by depositing a plant tissue structure on growth medium comprising an inducer, thereby exposing at least about 50% of the plant tissue structure (i.e. the site being in contact with the growth medium) to an inducing hydrogel. “Otherwise similar” is to be understood here as a using the same or similar hydrogel composition, the same or similar plant tissue and using otherwise (with the exception of the surface area of the plant tissue contacted with said hydrogel) the same or similar conditions.

In an aspect, provided is a hydrogel, preferably a hydrogel bead, comprising a compound. Preferably the hydrogel is an inductive hydrogel. An inductive hydrogel is understood herein as a hydrogel comprising a compound, wherein the compound is an inducer of gene expression. The inducer of gene expression can be a compound that disables a repressor of gene transcription or binds to an activator of gene transcription. Preferably, the inducer of gene expression binds, and consequently activates, an activator of gene transcription.

The inducer may be an activator of gene expression, preferably a (chemical) inducer of gene expression. Preferably, the (chemical) inducer interacts with, preferably binds to, a (chemically) responsive transcription factor, also indicated herein as a trans-activator. This interaction results in activation of gene transcription, preferably by binding of the transcription factor to an element controlling the expression of a gene.

The skilled person is aware of suitable responsive transcription factors or combinations of an inducer and responsive transcription factor (inducible systems). Suitable combinations are e.g. disclosed in Omelina, E. S. et al (Optogenetic and Chemical Induction Systems for Regulation of Transgene Expression in Plants: Use in Basic and Applied Research, Int. J. Mol. Sci. (2022), 23, 1737) and Moore I. et al (Transactivated and chemically inducible gene expression in plants, The Plant Journal (2006) 45, 651-683), which are incorporated herein by reference.

Suitable inducible systems include, but are not limited to, a Tet-derepressible system, a Tet-off system, a GVG/UAS system, a pOp6/LhGR system, β-estradiol (E2) induction system, a XVE system, a TGV system, an insecticide-induced system, a copper-induced system, and an ethanol-induced system. These systems are shown in Table 1 below, in addition to a suitable inducer and responsive transcription factor.

TABLE 1
Inducible gene expression systems

| | | Responsive |
|---|---|---|
| | | transcription |
| | | factor (trans- |
| System | Inducer | activator) |
| Tet-derepressible system | Tetracycline | tetR repressor |
| | | protein |
| Tet-off system | Tetracycline | tetR-VP16 |
| GVG/UAS system | Dexamethasone | GVG |
| pOp6/LhGR system | Dexamethasone | LhGR |
| β-estradiol (E2) | β-estradiol | ER-C1 |
| induction system | | |
| XVE system | β-estradiol | XVE |
| insecticide-induced system | Tebufenozide, | GVEcR |
| GVGE, GVE, VGE | methoxyfenozide | |
| copper-induced system | Copper | ACE1 |
| ethanol-induced system | Ethanol, acetaldehyde | AlcR |
| TGV system | Combination Tetracycline | TGV |
| | and Dexamethasone | |

The inducer may be an antibiotic, preferably tetracycline (Tet) or a derivative thereof. A preferred derivative is doxycycline. Tetracycline, and/or a derivative thereof, can be used as an inducer in the Tet-derepressible system and/or an inhibitor in the Tet-off system. Tet-dependent promoters can be developed by placing a Tet response element (TRE) consisting of the tetracycline operator (tetO) sequence repeats upstream of a promoter, preferably a minimal promoter. In the Tet-derepressible system, in the absence of tetracycline or a derivative thereof the tetR repressor binds TRE sequences, preventing the promoter from transcription initiation. Upon Tet addition, the tetR protein binds the Tet molecule and separates from TRE, thus, releasing gene expression.

In the Tet-off system, TRE is recognized by the tetR protein fused to the VP16 activation domain. In the absence of Tet, the chimeric tetR-VP16 protein binds TRE, activating the gene transcription. Upon Tet addition, the tetR-VP16 protein cannot bind to TRE, thus, the gene is not transcribed. Hence Tet may function as an inhibitor of gene expression and the hydrogel provided herein may comprise an inhibitor of gene expression, optionally a combination of an inhibitor of gene expression and an activator of gene expression. In the Tet-inducible systems, expression of the genes can be regulated by Tet and its derivatives, such as, but not limited to, doxycycline (Omelina et al, supra).

Preferably, the inducer is a compound that is capable of passively crossing the cell membrane. Preferably the inducer is a lipophilic compound. The inducer may be a steroid, preferably dexamethasone, β-estradiol, or a derivative thereof. The steroid is preferably used in combination with a responsive transcription factor (trans-activator), wherein the transcription factor is a fusion between a DNA-binding domain, an activation domain and a steroid receptor regulatory domain.

The inducer dexamethasone, or a derivative thereof, may interact with a trans-activator, wherein the trans-activator comprises a GAL4 DNA binding domain, a VP16 (activation) domain and a glucocorticoid receptor (GR) domain (GVG). Such GVG systems are well-known in the art and e.g. described in Moore et al, supra (2006) and Omelina et al, supra. Upon interaction with a steroid, such as e.g. dexamethasone or a derivative thereof, the trans-activator can initiate transcription upon binding one or more UAS elements, which elements are preferably linked to a (minimal) promoter, such as, but not limited to, the 35S minimal promoter. Optionally, at least about four or five UAS elements located upstream of the promoter.

Alternatively or in addition, the inducer dexamethasone or a derivative thereof may interact with a trans-activator, wherein the trans-activator LhGR comprises a domain binding to a LacOp element, preferably a DNA binding mutant lacl His17 DNA binding domain. Preferably, the trans-activator further comprises a GR domain and a GAL4 activation domain. Such LacOp systems are well-known in the art and e.g. described in Moore et al supra and Omelina et al, supra. Upon interaction of the trans-activator with a steroid, such as e.g. dexamethasone or a derivative thereof, the trans-activator can initiate transcription upon binding one or more LacOp elements, which elements are preferably linked to a minimal promoter, such as the 35S minimal promoter. Optionally at least about five or six LacOp elements are located upstream of the promoter.

Optionally the promoter comprising one or more binding sites for binding the trans-activator can be non-specific, tissue-specific or cell type-specific promoter. Coupling an inducible system as defined herein with a tissue- or cell type-specific promoter can result in inducible and tissue- or cell type-specific expression.

The inducer may be β-estradiol, or a derivative thereof. De inducer β-estradiol, or a derivative thereof, preferably binds to a chimeric ER-C1 protein or XVE protein to induce transcription. The trans-activator ER-C1 comprises a fusion between the activation domain of corn activator C1 and the estrogen receptor (ER). Upon interaction with β-estradiol or a derivative thereof, ER-C1 can bind to ER elements (ERE), which may be located upstream of a 35S minimal promoter. The chimeric XVE protein comprises a DNA binding domain from the bacterial LexA protein, the VP16 (activation) domain and an estrogen receptor ligand binding domain. Upon interaction with β-estradiol, or derivative thereof, the trans inducer may bind to one or more LexA operator sequence (OlexA) and activates gene expression. There is preferably more than one OlexA sequence located upstream of a promoter, preferably upstream of a minimal promoter, preferably upstream of a 35S minimal promoter. Optionally at least 5, 6, 7, or 8 copies of the OlexA are located upstream of the (minimal) promoter.

The inducer may be ethanol. Ethanol may interact with the trans-activator AlcR. Upon interaction, AlcR binds to the promoter sequences of the AlcA gene, which sequences can e.g. be fused to a minimal promoter, as e.g. described in Moore et al, supra and Omelina et al, supra.

The inducer may be an insecticide, preferably at least one of tebufenozide and methoxyfenozide. The insecticide may interact with a trans-activator to activate gene expression, wherein preferably the trans-activator is based on a chimeric GVEcR protein, comprising a glucocorticoid receptor activation domain and DNA binding domain, the VP16 activation domain and a worm ecdysone receptor ligand binding domain, or a variant thereof (e.g. GVGE (Gal4-VP16-GR-EcR), GVE (Gal4-VP16-EcR) or VGE (VP16-Gal4-EcR)) as e.g. described in Moore et al, supra and Omelina et al, supra.

The inducer may be a metal, preferably copper. Copper may bind to a trans-activator comprising the yeast copper-regulated transcription factor ACE1 fused to the VP16 activation domain. Following interaction with copper, the trans-activator can bind to a metal responsive element (MRE) linked to a, preferably minimal, promoter to induce transcription, such as e.g. described in Omelina et al, supra.

The hydrogel provided herein may comprise a combination of two or more inducers. Each one of the inducers may regulate the expression of a gene, by interaction with the respective trans-activator. Preferably, the hydrogel comprises a combination of steroids, preferably a combination of dexamethasone and β-estradiol, or derivatives thereof.

Also provided is a combination of hydrogels as defined herein, wherein preferably the combination of hydrogels used in the method provided herein. The combination of hydrogels may e.g. be applied to different parts of the same plant tissue or may be applied sequentially to the same tissue. The skilled person knows how to select a suitable combination of hydrogels. As a non-limiting example, a hydrogel may comprise dexamethasone, or a derivative thereof, and a second hydrogel may comprise β-estradiol, or a derivative thereof. Alternatively or in addition a first hydrogel may comprise tetracycline, or a derivative thereof and second hydrogel may comprise dexamethasone, or a derivative thereof, e.g. for use in the TGV system. The dual-controlled TGV system based on the combination of dexamethasone-inducible and Tetracycline-dependent expression system. A chimeric TGV protein comprises the tetR DNA binding domain, the GR ligand binding domain and the VP16 activation domain. In the absence of dexamethasone, TGV is inactive due to binding to the Hsp90 protein. Upon dexamethasone addition, TGV separates from Hsp90, interacts with a promoter and activates expression. The promoter further comprises one or more, preferably e.g. seven, repeats of the tetracycline operator (tetO) sequence. Upon interaction with tetracycline, the TGV protein dissociates again from the promoter, reducing expression. The TGV system is e.g. further described in Omelina et al, supra.

The hydrogel provided herein may comprise one or more further compounds, preferably one or more biological compounds. The skilled person readily understands that additional and/or combinations of compounds may be comprised in a hydrogel for use in a method as described herein. Such further compound may be relevant and/or serve in the process of regeneration and/or shoot production of the method of the invention. In addition or alternatively, the such further compound may serve another purpose, such as, but not limited to, the induction of (germline) mutations (e.g. programmed nucleases), gene insertions (e.g. transgenes) and/or selection (e.g. selection markers).

Preferably the compound is capable of diffusing out and/or through the hydrogel. Said further compound preferably is a lipophilic, hydrophilic or amphiphilic compound. Preferably, said further compound is lipophilic. Preferably the further compound is a compound that enters cells via passive diffusion through the lipid bilayer of the cell membrane. The further compound can be a biologically active compound or a non-biologically active compound. A biologically active compound refers to a compound that exerts a biological or chemical change in an organism or cell thereof, including but not limited to mammalians, vascular plants, non-vascular plants (eukaryotic algae, bryophytes), fungi, yeast and prokaryotic organisms (bacteria, cyanobacteria, etc.).

Preferably, the further compound is selected from the group consisting of an inducer of gene expression, a plant hormone, a peptide, an enzyme, a guide RNA, a (viral) vector, a bacterium, an oligonucleotide, a nucleic acid, a protein, or any combination thereof.

The further compound can be BSA (bovine serum albumin), GFP (green fluorescent protein), DNA, an antisense RNA or messenger RNA. The further compound can be an enzyme, e.g. selected from the group consisting of carbohydrases (including cellulases, amylases, pectinases and lactases), proteases, lipases, phytases, laccases, polymerases and nucleases.

A preferred further compound is at least one of an inducer of gene expression, a plant hormone, a guide RNA, a site-specific endonuclease, a (pre-)mRNA, a (viral) vector and a bacterium, or any combination thereof.

A preferred further compound comprised in the hydrogel is a plant hormone. Plant hormones (also known as phytohormones or ‘plant growth substances’) are chemicals that can regulate plant growth and development. A plant hormone can affect at least one of plant shape, seed growth, flowering time, the sex of flowers, senescence of leaves, and senescence of fruits. Alternatively or in addition, plant hormone can affect at least one of upward growth of tissues, downward growth of tissues, leaf formation, stem growth, fruit development, fruit ripening, plant longevity, and plant death.

The pant hormone may be selected from the group consisting of auxin, cytokinin, gibberellin, abscisic acid (ABA), ethylene, brassinosteroid, polyamine, salicylic acid, jasmonate, a plant peptide hormone, a polyamine, nitric oxide, strigolactones, Karrikins and triacontanol. A preferred plant hormone is a cytokinin or an auxin. Optionally, the hydrogel comprises more than one plant hormone, e.g. 2, 3, 4, 5 or more plant hormones. Optionally, the hydrogel comprises a combination of a cytokinin and an auxin, or multiple cytokines and/or multiple auxins.

The plant hormone comprised in the hydrogel can be an auxin. Auxins are a class of plant hormones that can have morphogen-like characteristics. The auxin can be an endogenously synthesized auxin. The endogenously synthesized auxin can be selected from the group consisting of indole-3-acetic acid (IAA), 4-chloroindole-3-acetic acid, phenylacetic acid, indole-3-butyric acid and indole-3-propionic acid. The auxin can be a synthetic auxin, e.g. an auxin analog. The synthetic auxin can be at least one of 1-naphthaleneacetic acid, 2,4-dichlorophenoxyacetic acid (2,4-D), α-Naphthalene acetic acid (α-NAA), 2-Methoxy-3,6-dichlorobenzoic acid (dicamba), 4-Amino-3,5,6-trichloropicolinic acid (tordon or picloram), 1-naphthaleneacetic acid (NAA), indole-3-butyric acid (IBA) and 2,4,5-trichlorophenoxyacetic acid (2,4,5-T). The auxin can be 1-naphthaleneacetic acid (NAA). In addition or alternatively, the plant hormone comprised in the hydrogel can be a gibberellin. The gibberellin can be a 19-carbon gibberellin or a 20-carbon gibberellin. The gibberellin can be a dihydroxylated gibberellin. The gibberellin can be at least one of GA1, GA3, GA4 and GA7.

The plant hormone comprised in the hydrogel can be a cytokinin. The cytokinin may be an adenine-type cytokinin or a phenylurea-type cytokinin. Similarly, the cytokinin can be a naturally produced phytohormone or can be a synthesized compound. The adenine-type cytokinin can be a plant hormone normally synthesized in at least one of roots, seeds and fruits. In addition, cambium and other actively dividing tissues can also synthesize cytokinins A non-limiting example of a naturally occurring adenine-type cytokinin is Zeatin as well as its metabolic precursor 2-isopentenyladenine (2iP). Non-limiting examples of synthetic adenine-type cytokinins are kinetin and 6-benzylaminopurine (BAP). Substituted urea compounds, such as thidiazuron and CPPU do not occur in plants but can act as cytokinins in tissue culture. The adenine-type cytokinin can be selected from the group consisting of kinetin, zeatin, trans-zeatin, cis-zeatin, dihydrozeatin, 6-benzylaminopurine and 2iP, and combinations thereof. The phenylurea-type cytokinin can be diphenylurea or thidiazuron.

The further compound comprised in the hydrogel can be a vector. A preferred expression vector is a naked DNA, a DNA complex or a viral vector. A preferred naked DNA is a linear or circular nucleic acid molecule, e.g. a plasmid. A plasmid refers to a circular double stranded DNA loop into which additional DNA segments can be inserted, such as by standard molecular cloning techniques. A DNA complex can be a DNA molecule coupled to any carrier suitable for delivery of the DNA into the cell. A preferred carrier is selected from the group consisting of a lipoplex, a liposome, a polymersome, a polyplex, PEG, a dendrimer, an inorganic nanoparticle, a virosome and cell-penetrating peptides. The vector is preferably a viral vector. Preferably, the viral vector is a vector suitable of infecting the plant cell. Preferably, the viral vector is at least one of a Tobacco Rattle Virus (TRV), a Bean yellow dwarf virus (BeYDV), a Cabbage leaf curl virus (CaLCuV), a tobravirus and a Wheat dwarf virus (WDV). Optionally, the viral vector comprises a transgene for expression in the plant cell

The further compound can be a bacterium. Preferably, the bacterium is an Agrobacterium, preferably an Agrobacterium tumefaciens. Optionally, the bacterium is modified to comprise a transgene for expression in the plant cell.

The further compound comprised in the hydrogel can be a guide RNA, preferably a guide RNA that directs a CRISPR-protein to location within the plant genome. The guide RNA may be suitable for e.g. directing a Cas9 protein, a Cpf1 protein or a Mad7 protein to a location in the plant genome. In this embodiment, at least part of the cells of the plant tissue may comprise a sequence encoding a CRISPR-protein, preferably at least one of a Cas9 (SpCas9, StCas9, SaCas9, ScCas9, and dead Cas9), CasX, CasY, Cas-Phi, Cas12a (Cpf1), Cas13, Cas14 and MAD7. The guide RNA comprised in the hydrogel may be a crRNA (e.g. in the case of Cpf1), a combination of a crRNA and a tracrRNA, or a single guide RNA (e.g. in the case of a Cas9 protein).

The further compound can be a gene modifying molecule such as, but not limited to, a gene silencing molecule, such as an siRNA and/or a miRNA, a gene replacement and/or a gene insertion molecule or a molecule that generates targeted modifications in the genome, or a messenger RNA encoding such molecule. Optionally, said miRNA or siRNA may be targeting the RBR RNA transcript. A mature siRNA or miRNA can comprise at least 20, 21, 22, 23, 24 or at least 25 contiguous nucleotides. The mature siRNA or miRNA can comprise at least 20, 21, 22, 23, 24 or at least 25 contiguous nucleotides that that have at least about 95%, 96%, 97%, 98%, 99% or 100% sequence complementarity with a contiguous sequence in the endogenous RBR transcript. The endogenous RBR transcript is preferably the endogenous RBR mRNA molecule, and preferably includes the 3′ and 5′ untranslated RBR sequence. Hence, the sequence of the non-coding small RNA can be, partly or completely, complementary to a sequence comprised in the RBR coding sequence or complementary to a sequence comprised in the 3′r 5′ntranslated region of the RBR transcript. Preferably, the siRNA or miRNA can be partly or completely complementary to a sequence comprised in the RBR coding sequence. For example, at least 20, 21, 22, 23, 24 or at least 25 contiguous nucleotides of the small RNA molecule has at least about 80%, 85%, 90%, 95%, 96%, 97%, 98%, 99% or 100% sequence complementarity with a contiguous sequence of at least 20, 21, 22, 23, 24 or at least 25contiguous nucleotides of the endogenous RBR transcript, respectively. The skilled person understands how to design a small RNA molecule that is capable of downregulating endogenous RBR protein expression using conventional RNAi, wherein the RBR protein is a RBR protein as defined herein above. Optionally, the small RNA molecule for inhibiting RBR expression can comprise a sequence having at least about 85%, 90%, 95%, 96%, 97%, 98%, 99% or 100% sequence identity with SEQ ID NO: 34 (RBR miRNA precursor). The small RNA molecule can comprise a sequence having at least about 85%, 90%, 95%, 96%, 97%, 98%, 99% or 100% sequence identity with SEQ ID NO: 35 (RBR mature miRNA).

The further compound can be a molecule that generates a targeted modification (targeted mutagenesis) in the genome, such as a CRISPR ribonucleoprotein (RNP), zinc finger nuclease (ZFN), transcription activator-like effector nuclease (TALEN), and oligonucleotides (such as small interfering RNA (siRNA), and short hairpin RNA (shRNA)) and/or an mRNA encoding the a programmable nuclease such as a CRISPR endonuclease and/or a TALEN. The targeted modification is preferably in a sequence of interest. In some embodiments, the further compound is a CRISPR protein or a DNA or RNA molecule encoding a CRISPR protein. The CRISPR-protein may be at least one of a Cas9 (SpCas9, StCas9, SaCas9, ScCas9, and dead Cas9), CasX, CasY, Cas-Phi, Cas12a (Cpf1), Cas13, Cas14 and MAD7. The further compound can be a complex of a CRISPR-protein and a guide RNA.

The further compound can be a messenger RNA (mRNA) or pre-mRNA. The protein encoded by the (pre-)mRNA preferably is at least one of an enzyme, a hormone and a transcription factor. Optionally, the (pre-)mRNA encodes a CRISPR-protein, preferably a CRISPR-protein as defined herein.

A hydrogel is a three-dimensional, hydrophilic network. The hydrogel may be formed by gelling of a polymer solution, preferably in the presence of a compound as defined herein. The terms “gelling” and “gelation” may be used interchangeably herein. During gelling, crosslinks are formed between the polymers comprised in the polymer solution. Said crosslinks may comprise covalent bonds, ionic bonds, molecular entanglements, hydrogen bonding, hydrophobic interactions, van der Waals forces and/or dipole-dipole interactions. Preferably, said crosslinks comprise covalent bonds and/or ionic bonds. The nature of these crosslinks is primarily determined by the chemical structure of the polymers and optionally of the compound as defined herein. For example, a solution comprising a polymer with ionic pendant groups may form ionic bonds upon gelling. A crosslink may comprise one or more linking agents such as ions.

Gelling occurs preferably under specific conditions, which may be characterized by one or more parameters such as temperature, pH, the concentration of specific ions, and the presence of gelation inducers, alone or in combination. A polymer solution may have a lower critical gelation temperature (LCGT), around which gelling will occur upon heating. A polymer solution may have an upper critical gelation temperature (UCGT), around which gelling will occur upon cooling. Besides temperature, a specific condition for inducing gelling may include the concentration of specific ions. For example, gelling an aqueous solution of alginate occurs in the presence of e.g. calcium ions. Without wishing to be bound by any theory, it is generally considered that the calcium ions allow the formation of ionic crosslinks between the carboxylic pendant groups of the alginate chains, as the ions act as linking agent.

Preferably, a hydrogel provided herein is formed from a, preferably aqueous, polymer solution comprising an ionic polymer, preferably an anionic polymer, preferably an anionic polymer comprising carboxylic pendant groups, wherein said hydrogel comprises ionic crosslinks. Such ionic crosslinks comprise an ion, preferably calcium, as a linking agent. Hence preferably a hydrogel provided herein comprises cross-linked ionic polymers, preferably anionic polymers having carboxylic pendant groups.

In the context of this application, it is understood that an anionic polymer refers to a polymer which is negatively charged under the conditions present in the corresponding polymer solution, notwithstanding that said compound or group may be neutral or positively charged under different conditions. A corresponding definition holds for neutral and cationic polymers.

Preferably a hydrogel provided herein is formed from a, preferably aqueous, polymer solution comprising a polysaccharide or a derivative thereof. More preferably, said polysaccharide or derivative thereof comprises one or more sugar acids, preferably uronic acids. Preferably, a hydrogel provided herein is formed from a polymer solution comprising alginate or a derivative thereof. Hence preferably a hydrogel provided herein comprises cross-linked polysaccharides or derivatives thereof, preferably wherein said polysaccharides or derivatives thereof comprise an uronic acid. Preferably, the hydrogel provided herein comprises alginate or a derivative thereof.

Alginate is the family of linear copolymers of (1,4)-linked β-D-mannuronate (M) and α-L-guluronate (G) residues or monomers. Said M and G monomers are arranged as consecutive G residues, consecutive M residues or alternating M and G residues in alginate. The ratio of the number of M and the number of G residues, and the lengths of the blocks of G residues, the blocks of M residues and the blocks of MG residues in an alginate is dependent on the source from which said alginate is obtained. A preferred source of alginate is from a species within the class of Phaeophycea (brown algea). The alginate may be obtainable from a species of at least one of Macrocystis, Sargassum and Laminaria. Preferably, the Laminaria is at least one of Laminaria digitate, Laminaria hyperborea and Laminaria durvillaea.

Preferably, the G/M ratio is ≥1.5. Preferably, the M/G ratio is at least about 0.7 or 0.8, preferably about 0.7-1.4 or about 0.8-1.6.

The viscosity of the alginate when dissolved in water may be high, medium or low. Preferably the viscosity (mPa/s) is about 4-12 or about 20-200. Preferred alginate derivatives are amphiphilic alginates and alginates covalently attached to oligopeptides. Amphiphilic alginates are derivable from alginates by covalently attaching hydrophobic groups such as alkyls to the carboxylic pendant groups.

Preferably a compound, preferably a compound as defined herein, preferably an inducer as defined herein, is added to the polymer solution prior to gelling, such that compound is captured within the hydrogel upon gelling. Preferably said inducer is captured homogenously within said hydrogel. The compound is preferably capable of diffusing out the formed hydrogel.

Further provided is a hydrogel bead, wherein the bead comprises a compound, preferably a compound as defined herein, preferably an inducer as defined herein. The bead may comprise a uniform distribution of the compound. The hydrogel bead can be applied onto a plant tissue. After applying the hydrogel bead onto the plant tissue, the compound can at least partly diffuse out of the hydrogel and forms a gradient in the plant tissue. An exemplary approach is also outlined in the example section below. Optionally, provided is a single hydrogel bead. Alternatively, provided is a combination of 2 or more hydrogel beads. The two or more beads may comprise the same compound, preferably a compound as defined herein, or may comprise different compounds, preferably a combination of different compounds as defined herein. Alternatively or in addition, the concentration of the, optionally different, compounds may vary between the beads or the combination of beads. Optionally, provided are combination of at least 2, 3, 4, 5, 6, 7, 8, 9, 10, 50, 100, 200, 300, 400, 500, 1000, 5000, 10000 or more hydrogel beads.

The size of the hydrogel bead may vary, e.g. between about 0.1 nm-100 mm. Preferably, the bead is a macrosphere, microsphere or nanosphere. The macrosphere preferably has a diameter of about 0.1 mm-100 millimetre (mm), preferably 0.1 mm-10 mm, the microsphere preferably has a diameter of about 0.1 μm-100 micrometre (μm), preferably 0.1 μm-10 μm, and the nanosphere preferably has a diameter of about 0.1 nm-100 nanometre (nm), preferably 0.1 nm-10 nm.

The hydrogel bead is preferably a macrosphere, wherein the diameter of the macrosphere is preferably about 0.1 mm-100 mm, 0.5 mm-70 mm, 1 mm-50 mm, 2 mm-10 mm, 3 mm-8 mm, or about 4 mm-6 mm. Preferably, the diameter of the macrosphere is about 0.1 mm, 0.5 mm, 1 mm, 2 mm, 3 mm, 4 mm, 5 mm, 10 mm, 20 mm, 30 mm, 40 mm, 50 mm, 70 mm or about 100 mm. The hydrogel bead is preferably a microsphere, wherein the diameter of the microsphere is preferably about 0.1 μm-100 μm, 0.5 μm-70 μm, 1 μm-50 μm, 2 μm-10 μm, 3 μm-8 μm, or about 4 μm-6 μm. Preferably, the diameter of the microsphere is about 0.1 μm, 0.5 μm, 1 μm, 2 μm, 3 μm, 4 μm, 5 μm, 10 μm, 20 μm, 30 μm, 40 μm, 50 μm, 70 μm or about 100 μm. The hydrogel bead is preferably a nanosphere, wherein the diameter of the nanosphere is preferably about 0.1 nm-100 nm, 0.5 nm-70 nm, 1 nm-50 nm, 2 nm-10 nm, 3 nm-8 nm, or about 4 nm-6 nm. Preferably, the diameter of the microsphere is about 0.1 nm, 0.5 nm, 1 nm, 2 nm, 3 nm, 4 nm, 5 nm, 10 nm, 20 nm, 30 nm, 40 nm, 50 nm, 70 nm or about 100 nm.

The concentration of the compound as described herein, preferably the inducer as defined herein, in the hydrogel is a concentration that provides an effect in one or more cells of the plant tissue. Hence the concentration of the compound is an effective concentration. Preferably the concentration of the inducer results in inducing an inducible promoter, optionally upon binding to a trans-activator comprised in the plant tissue.

The concentration of the compound, preferably the inducer, in the hydrogel, preferably in the hydrogel bead can be about 0.1 μM-100 mM, preferably about 0.1 mM-100 mM, 0.1 μM-100 μM, 0.1 nM-100 nM or about 0.1 μM-100 μM.

The concentration of the compound, preferably the inducer, in the hydrogel may be about 0.1 mM-100 mM, 0.5 mM-70 mM, 1 mM-50 mM, 2 mM-10 mM, 3 mM-8 mM, or about 4 mM-6 mM. Preferably, the concentration of the compound can be about 0.1 mM, 0.5 mM, 1 mM, 2 mM, 3 mM, 4 mM, 5 mM, 10 mM, 20 mM, 30 mM, 40 mM, 50 mM, 70 mM or about 100 mM. The concentration of the compound in the hydrogel may be about 0.1 μM-100 μM, 0.5 μM-70 μM, 1 μM-50 μM, 2 μM-10 μM, 3 μM-8 μM, or about 4 μM-6 μM. Preferably, the concentration of the compound can be about 0.1 μM, 0.5 μM, 1 μM, 2 μM, 3 μM, 4 μM, 5 μM, 10 μM, 20 μM, 30 μM, 40 μM, 50 μM, 70 μM or about 100 μM. The concentration of the compound in the hydrogel may be about 0.1 nM-100 nM, 0.5 nM-70 nM, 1 nM-50 nM, 2 nM-10 nM, 3 nM-8 nM, or about 4 nM-6 nM. Preferably, the concentration of the compound can be about 0.1 nM, 0.5 nM, 1 nM, 2 nM, 3 nM, 4 nM, 5 nM, 10 nM, 20 nM, 30 nM, 40 nM, 50 nM, 70 nM or about 100 nM. The concentration of the compound in the hydrogel may be about 0.1 pM-100 pM, 0.5 pM-70 pM, 1 pM-50 pM, 2 pM-10 pM, 3 pM-8 pM, or about 4 pM-6 pM. Preferably, the concentration of the compound can be about 0.1 pM, 0.5 pM, 1 pM, 2 pM, 3 pM, 4 pM, 5 pM, 10 pM, 20 pM, 30 pM, 40 pM, 50 pM, 70 pM or about 100 pM.

A hydrogel is a three-dimensional, hydrophilic network. As understood herein, an oil or a fat is thus not an hydrogel. Lanolin, or wool wax, is not a hydrogel as defined herein.

In an aspect, provided is a method for producing a plant structure, comprising the steps of:
a) providing a plant tissue;
b) locally contacting the plant tissue to an inductive hydrogel comprising a compound, wherein the compound is an inducer of gene expression; and
c) producing a plant structure, preferably a plant shoot.

In step a) of the method provided herein, a plant tissue is provided. The plant tissue may be of any plant species or genus. Preferably, the plant or plant cell is capable of regeneration, preferably capable of at least one of shoot organogenesis and somatic embryogenesis.

The plant tissue may be tissue from a monocotyledonous or dycotyledonous plant. The plant tissue may be from a crop or a grain plant. A non-limiting example of a grain plant for use in the method provided herein is cassava, corn, sorghum, soybean, wheat, oat or rice. A crop plant is a plant species which is cultivated and bred by humans. A crop plant may be cultivated for food and feed purposes (e.g. field crops), or for ornamental purposes (e.g. production of flowers for cutting, grasses for lawns, etc.). A crop plant as defined herein also includes plants from which non-food products are harvested, such as oil for fuel, plastic polymers, pharmaceutical products, cork and the like.

The plant tissue may be or be derived from a plant of that belongs to the Brassicaceae, Cucurbitaceae, Fabaceae, Gramineae, Solanaceae, Asteraceae (Compositae), Rosaceae or Poaceae. Optionally, the plant tissue is or is derived from a plant that is selected from the group consisting of maize/corn (Zea species), wheat (Triticum species), barley (e.g. Hordeum vulgare), oat (e.g. Avena sativa), sorghum (Sorghum bicolor), rye (Secale cereale), soybean (Glycine spp, e.g. G. max), cotton (Gossypium species, e.g. G. hirsutum, G. barbadense), Brassica spp. (e.g. B. napus, B. juncea, B. oleracea, B. rapa, etc), sunflower (Helianthus annuus), safflower, yam, cassava, alfalfa (Medicago sativa), rice (Oryza species, e.g. O. sativa indica cultivar-group or japonica cultivar-group), forage grasses, pearl millet (Pennisetum spp. e.g. P. glaucum), tree species (Pinus, poplar, fir, plantain, etc), tea, coffea, oil palm, coconut, vegetable species, such as pea, zucchini, beans (e.g. Phaseolus species), hot pepper, cucumber, artichoke, asparagus, eggplant, broccoli, garlic, leek, lettuce, onion, radish, turnip, tomato, potato, Brussels sprouts, carrot, cauliflower, chicory, celery, spinach, endive, fennel, beet, fleshy fruit bearing plants (grapes, peaches, plums, strawberry, mango, apple, plum, cherry, apricot, banana, blackberry, blueberry, citrus, kiwi, figs, lemon, lime, nectarines, raspberry, watermelon, orange, grapefruit, etc.), ornamental species (e.g. Rose, Petunia, Chrysanthemum, Lily, Gerbera species), herbs (mint, parsley, basil, thyme, etc.), woody trees (e.g. species of Populus, Salix, Quercus, Eucalyptus), fibre species e.g. flax (Linum usitatissimum) and hemp (Cannabis sativa). The plant tissue may also be from a tree or production plant, fruit or vegetable (e.g., trees such as citrus trees, e.g., orange, grapefruit or lemon trees; peach or nectarine trees; apple or pear trees; nut trees such as almond or walnut or pistachio trees; nightshade plants; plants of the genus Brassica; plants of the genus Lactuca; plants of the genus Spinacia; plants of the genus Capsicum; plants of the genus Solanum, preferably Solanum lycopersicum). Preferably, the plant is a plant of the genus Solanum. A preferred plant for use in the method provided herein is a Solanum lycopersicum or Capsicum annuum plant.

In another preferred embodiment, the plant tissue is from a plant that is selected from the group consisting of asparagus, barley, blackberry, blueberry, broccoli, cabbage, canola, carrot, cassava, cauliflower, chicory, cocoa, coffee, cotton, cucumber, eggplant, grape, hot pepper, lettuce, maize, melon, oilseed rape, pepper, potato, pumpkin, raspberry, rice, rye, sorghum, spinach, squash, strawberry, sugar cane, sugar beet, sunflower, sweet pepper, tobacco, tomato, water melon, wheat, and zucchini.

Optionally, the plant tissue is from a recalcitrant plant, wherein a recalcitrant plant is to be understood herein as a plant in which regeneration fails or is poor. Optionally, the recalcitrant plant or cell thereof fails to regenerate or shows a poor regeneration efficiency under conditions known in the art to be optimal for regeneration, such as, but not limited to, conditions that allow for regeneration in the presence of externally supplied growth regulators. The skilled person is aware of recalcitrant plants. Although within species both recalcitrant and regenerative cultivars, varieties and/or accessions may exist, in general, pepper, soybean and sugar beet are non-limiting examples of recalcitrant plants, and cells thereof are non-limiting examples of recalcitrant plant cells. Typical non-limiting examples of plants known in the art to be recalcitrant are pepper (Capsicum annuum), sugarbeet (Beta vulgaris, more in particular Beta vulgaris subsp. vulgaris), soybean (Gycine max), sunflower (Helianthus annuus), cotton (Gossipium hirsutum), hemp or cannabis (Cannabis sativa), strawberry (Fragaria×ananassa), hops (Humulus lupulus), melon (Cucumis melo) and cucumber (Cucumis sativus). However, the method of the invention can be applied to all plants or plant cells that in some circumstance benefit from an increase in regeneration efficiency. Non-limiting examples of workflows for which regeneration is a bottleneck are the general multiplication of (clonally propagated) plant material, especially in case of haploid plant material or genetically complex (e.g. highly ploidy and heterozygous) F1 populations, but also advanced plant biotech workflows such as, but not limited to, targeted plant genome editing, production of (stable or transient) transformants and doubled haploid induction. Therefore, the method of the invention may be part of such plant biotech workflows.

At least part of the cells of the plant tissue comprise an expression construct as defined herein, wherein said expression construct comprises sequence encoding a regeneration factor operably linked to an inducible promoter, and wherein said inducible promoter is activated by the inducer present in the inductive hydrogel. Optionally, the inducer is capable of inducing or activating the promoter after binding of the inducer to a trans-activator present in said at least part of the cells of the plant tissue. Said trans-activator may be present in the genome of these cells or on an expression construct. Optionally, said trans-activator is present on the expression construct as the construct comprising the sequence encoding a regeneration factor operably linked to an inducible promoter. Therefore, in a particular embodiment, the plant tissue of step a) comprises an expression construct comprising at least one sequence encoding a regeneration factor operably linked to an inducible promoter and a sequence encoding a trans-activator operably linked to a promoter that may be a promoter that is constitutively active in a plant cell comprising said construct.

The plant tissue comprising an expression construct as defined herein can be provided by introducing the expression construct into a plant cell, e.g. by Agrobacterium or viral transfection. Optionally, the plant tissue of step a) of the method provided herein is developed from said transfected plant cell. Optionally, the plant tissue of step a) is formed by (inducing the) formation of calli from said transfected plant cell. Preferably, the calli are formed by placing the transfected plant cell on CIM (callus inducible medium) and/or placing formed calli on SIM (shoot inducible medium). The components of CIM and SIM are well-known in the art and may vary per plant species used in the method provided herein.

The plant tissue of step a) of the method provided herein can be a differentiated or undifferentiated tissue including, but not limited to the following: roots, stems, shoots, leaves, pollen, seeds, tumor tissue, embryos and callus tissue. The plant tissue may be an (immature) embryo, a hypocotyl, a cotyledon, a leaf or a (decapitated) scion.

Preferably, at least part of the cells of the plant tissue of step a) of the method provided herein comprise an inducible promoter operably linked to at least one sequence encoding a regeneration factor, also indicated herein as morphogenic developmental polypeptide. The terms “regeneration factor”, “morphogenic developmental polypeptide” and “morphogenic polypeptide” can thus be used interchangeable herein. The inducible promoter may comprise one or more elements for binding of a (activated) trans-activator. The element(s) binding a trans-activator are preferably as defined herein. As non-limiting examples, the dexamethasone-bound GVG preferably binds to one or more UAS elements, preferably having at least 80%, 85%, 90%, 95%, 96%, 97%, 98%, 99% or 100% sequence identity with SEQ ID NO: 1, the dexamethasone-bound LhGR preferably binds to one or more LacOp elements, preferably having at least 80%, 85%, 90%, 95%, 96%, 97%, 98%, 99% or 100% sequence identity with SEQ ID NO: 2 and the β-estradiol-bound XVE preferably binds to one or more LexAop elements, preferably having at least 80%, 85%, 90%, 95%, 96%, 97%, 98%, 99% or 100% sequence identity with SEQ ID NO: 3, The elements are preferably linked to a minimal promoter, such as, but not limited to, the Cauliflower Mosaic Virus (CaMV) 35S promoter. Optionally, the elements are linked to a tissue-specific promoter.

Upon binding an inducer, the trans-activator binds and activates the inducible promoter, resulting in the expression of one or more morphogenic developmental polypeptides. As used herein, the term “morphogenic polypeptide” or “morphogenic developmental polypeptide” means a polypeptide that when ectopically expressed stimulates formation of a somatically-derived structure that can produce a plant. More precisely, ectopic expression of the morphogenic polypeptide stimulates the de novo formation of a somatic embryo or an organogenic structure, such as a shoot meristem, that can produce a plant. This stimulated de novo formation occurs either in the cell in which the morphogenic polypeptide is expressed, or in a neighbouring cell. A morphogenic polypeptide can be a transcription factor that regulates expression of other genes, or a polypeptide that influences hormone levels in a plant tissue, both of which can stimulate morphogenic changes.

Preferably, the morphogenic developmental polypeptide is at least one of a WUS/WOX homeobox polypeptide, a PLT (PLETHORA) protein, a polypeptide comprising two AP-2 DNA binding domains, WIND1. Preferably, the morphogenic developmental polypeptide having induced expression is at least one of a WUS/WOX homeobox polypeptide and a PLT protein. Preferably, the morphogenic developmental polypeptide having induced expression is at least one of a WOX5 homeobox polypeptide and a PLT1 protein. Preferably, the morphogenic developmental polypeptide having induced expression is the combination of a WOX5 homeobox polypeptide and a PLT1 protein. Preferably, the morphogenic developmental polypeptide having induced expression is the combination of a WOX5 homeobox polypeptide, a PLT1 protein and a WIND1 protein.

The WUS/WOX homeobox polypeptide is preferably selected from the group consisting of WUS1, WUS2, WUS3, WOX2A, WOX4, WOX5, WOX5A, or WOX9 polypeptide (see e.g. U.S. Pat. Nos. 7,348,468 and 7,256,322 and US Patent Application Publication Numbers 2017/0121722 and 2007/0271628, herein incorporated by reference in their entirety and van der Graaff et al., 2009, Genome Biology 10:248). The functional WUS/WOX homeobox polypeptide for use in the method provided herein can be obtained from or derived from any plant. A functional WUS/WOX polypeptide contains a homeobox DNA binding domain, a WUS box, and an EAR repressor domain useful in the methods of the disclosure and are listed in Table 1 of WO2020214986, in particular SEQ ID NO: 246-310 of WO2020214986, which sequences are incorporated herein by reference.

A preferred WUS/WOX homeobox polypeptide is WOX5. The amino acid sequence of the WOX5 protein can have at least about 50%, 55%, 60%, 65%, 70%, 75%, 80%, 85%, 90%, 95%, 98%, 99% or 100% sequence identity with SEQ ID NO: 4. SEQ ID NO: 4 is the Arabidopsis thaliana WOX5 protein. In an embodiment, the WOX5 amino acid sequence is or is derived from AT3G11260, a homolog thereof, or a sequence having at least about 50%, 55%, 60%, 65%, 70%, 75%, 80%, 85%, 90%, 95%, 98%, 99% or 100% sequence identity with AT3G11260 or its homolog. In an embodiment, the WOX5 protein is encoded by a nucleotide sequence having at least about 50%, 55%, 60%, 65%, 70%, 75%, 80%, 85%, 90%, 95%, 98%, 99% or 100% sequence identity with SEQ ID NO: 5. The nucleotide sequence encoding the WOX5 protein can be, or can be derived from the gene AT3G11260, a homolog thereof or a sequence having at least about 50%, 55%, 60%, 65%, 70%, 75%, 80%, 85%, 90%, 95%, 98%, 99% or 100% sequence identity with AT3G11260 or its homolog. The percentage identity can be determined over the full length of the genomic sequence. Alternatively the percentage identity can be determined over the full length of the coding sequence of the gene.

The PLT protein is preferably selected from the group consisting of PLT1, PLT2, PLT3, PLT4, PLT5 and PLT7. Preferably, the PLT protein is at least one of PLT1, PLT4 and PLT5. Preferably, the PLT protein is PLT1.

The amino acid sequence of the PLT1 protein can have at least about 50%, 55%, 60%, 65%, 70%, 75%, 80%, 85%, 90%, 95%, 98%, 99% or 100% sequence identity with SEQ ID NO: 6. SEQ ID NO: 6 is the Arabidopsis thaliana PLT1 protein. In an embodiment, the PLT1 amino acid sequence is or is derived from AT3G20840, a homolog thereof, or a sequence having at least about 50%, 55%, 60%, 65%, 70%, 75%, 80%, 85%, 90%, 95%, 98%, 99% or 100% sequence identity with AT3G20840 or its homolog. In an embodiment, the PLT1 protein is encoded by a nucleotide sequence having at least about 50%, 55%, 60%, 65%, 70%, 75%, 80%, 85%, 90%, 95%, 98%, 99% or 100% sequence identity with SEQ ID NO: 7. The nucleotide sequence encoding the PLT1 protein can be, or can be derived from the gene AT3G20840, a homolog thereof or a sequence having at least about 50%, 55%, 60%, 65%, 70%, 75%, 80%, 85%, 90%, 95%, 98%, 99% or 100% sequence identity with AT3G20840 or its homolog. The percentage identity can be determined over the full length of the genomic sequence. Alternatively the percentage identity can be determined over the full length of the coding sequence of the gene.

The amino acid sequence of the PLT2 protein can have at least about 50%, 55%, 60%, 65%, 70%, 75%, 80%, 85%, 90%, 95%, 98%, 99% or 100% sequence identity with SEQ ID NO: 8. SEQ ID NO: 8 is the Arabidopsis thaliana PLT2 protein. In an embodiment, the PLT2 amino acid sequence is or is derived from AT1G51190, a homolog thereof, or a sequence having at least about 50%, 55%, 60%, 65%, 70%, 75%, 80%, 85%, 90%, 95%, 98%, 99% or 100% sequence identity with AT1G51190 or its homolog. In an embodiment, the PLT2 protein is encoded by a nucleotide sequence having at least about 50%, 55%, 60%, 65%, 70%, 75%, 80%, 85%, 90%, 95%, 98%, 99% or 100% sequence identity with SEQ ID NO: 9. The nucleotide sequence encoding the PLT2 protein can be, or can be derived from the gene AT1G51190, a homolog thereof or a sequence having at least about 50%, 55%, 60%, 65%, 70%, 75%, 80%, 85%, 90%, 95%, 98%, 99% or 100% sequence identity with AT1G51190 or its homolog. The percentage identity can be determined over the full length of the genomic sequence. Alternatively the percentage identity can be determined over the full length of the coding sequence of the gene.

The amino acid sequence of the PLT3 protein can have at least about 50%, 55%, 60%, 65%, 70%, 75%, 80%, 85%, 90%, 95%, 98%, 99% or 100% sequence identity with SEQ ID NO: 10. SEQ ID NO: 10 is the Arabidopsis thaliana PLT3 protein. In an embodiment, the PLT3 amino acid sequence is or is derived from AT5G10510, a homolog thereof, or a sequence having at least about 50%, 55%, 60%, 65%, 70%, 75%, 80%, 85%, 90%, 95%, 98%, 99% or 100% sequence identity with AT5G10510 or its homolog. In an embodiment, the PLT3 protein is encoded by a nucleotide sequence having at least about 50%, 55%, 60%, 65%, 70%, 75%, 80%, 85%, 90%, 95%, 98%, 99% or 100% sequence identity with SEQ ID NO: 11. The nucleotide sequence encoding the PLT3 protein can be, or can be derived from the gene AT5G10510, a homolog thereof or a sequence having at least about 50%, 55%, 60%, 65%, 70%, 75%, 80%, 85%, 90%, 95%, 98%, 99% or 100% sequence identity with AT5G10510 or its homolog. The percentage identity can be determined over the full length of the genomic sequence. Alternatively the percentage identity can be determined over the full length of the coding sequence of the gene.

The amino acid sequence of the PLT4 protein can have at least about 50%, 55%, 60%, 65%, 70%, 75%, 80%, 85%, 90%, 95%, 98%, 99% or 100% sequence identity with SEQ ID NO: 12. SEQ ID NO: 12 is the Arabidopsis thaliana PLT4 protein. In an embodiment, the PLT4 amino acid sequence is or is derived from AT5G17430, a homolog thereof, or a sequence having at least about 50%, 55%, 60%, 65%, 70%, 75%, 80%, 85%, 90%, 95%, 98%, 99% or 100% sequence identity with AT5G17430 or its homolog. In an embodiment, the PLT4 protein is encoded by a nucleotide sequence having at least about 50%, 55%, 60%, 65%, 70%, 75%, 80%, 85%, 90%, 95%, 98%, 99% or 100% sequence identity with SEQ ID NO: 13. The nucleotide sequence encoding the PLT4 protein can be, or can be derived from the gene AT5G17430, a homolog thereof or a sequence having at least about 50%, 55%, 60%, 65%, 70%, 75%, 80%, 85%, 90%, 95%, 98%, 99% or 100% sequence identity with AT5G17430 or its homolog. The percentage identity can be determined over the full length of the genomic sequence. Alternatively the percentage identity can be determined over the full length of the coding sequence of the gene.

The amino acid sequence of the PLT5 protein can have at least about 50%, 55%, 60%, 65%, 70%, 75%, 80%, 85%, 90%, 95%, 98%, 99% or 100% sequence identity with SEQ ID NO: 14. SEQ ID NO: 14 is the Arabidopsis thaliana PLT5 protein. In an embodiment, the PLT5 amino acid sequence is or is derived from AT5G57390, a homolog thereof, or a sequence having at least about 50%, 55%, 60%, 65%, 70%, 75%, 80%, 85%, 90%, 95%, 98%, 99% or 100% sequence identity with AT5G57390 or its homolog. In an embodiment, the PLT5 protein is encoded by a nucleotide sequence having at least about 50%, 55%, 60%, 65%, 70%, 75%, 80%, 85%, 90%, 95%, 98%, 99% or 100% sequence identity with SEQ ID NO: 15. The nucleotide sequence encoding the PLT5 protein can be, or can be derived from the gene AT5G57390, a homolog thereof or a sequence having at least about 50%, 55%, 60%, 65%, 70%, 75%, 80%, 85%, 90%, 95%, 98%, 99% or 100% sequence identity with AT5G57390 or its homolog. The percentage identity can be determined over the full length of the genomic sequence. Alternatively the percentage identity can be determined over the full length of the coding sequence of the gene.

The amino acid sequence of the PLT7 protein can have at least about 50%, 55%, 60%, 65%, 70%, 75%, 80%, 85%, 90%, 95%, 98%, 99% or 100% sequence identity with SEQ ID NO: 16. SEQ ID NO: 16 is the Arabidopsis thaliana PLT7 protein. In an embodiment, the PLT7 amino acid sequence is or is derived from AT5G65510, a homolog thereof, or a sequence having at least about 50%, 55%, 60%, 65%, 70%, 75%, 80%, 85%, 90%, 95%, 98%, 99% or 100% sequence identity with AT5G65510 or its homolog. In an embodiment, the PLT7 protein is encoded by a nucleotide sequence having at least about 50%, 55%, 60%, 65%, 70%, 75%, 80%, 85%, 90%, 95%, 98%, 99% or 100% sequence identity with SEQ ID NO: 17. The nucleotide sequence encoding the PLT7 protein can be, or can be derived from the gene AT5G65510, a homolog thereof or a sequence having at least about 50%, 55%, 60%, 65%, 70%, 75%, 80%, 85%, 90%, 95%, 98%, 99% or 100% sequence identity with AT5G65510 or its homolog. The percentage identity can be determined over the full length of the genomic sequence. Alternatively the percentage identity can be determined over the full length of the coding sequence of the gene.

The polypeptide comprising two AP-2 DNA binding domains is preferably a polypeptide selected from the group consisting of an ODP2, BBM2, BMN2, or BMN3 polypeptide. The amino acid sequence of the ODP2 protein can have at least about 50%, 55%, 60%, 65%, 70%, 75%, 80%, 85%, 90%, 95%, 98%, 99% or 100% sequence identity with any one of SEQ ID NO: 18. In an embodiment, the OPD2 protein is encoded by a nucleotide sequence having at least about 50%, 55%, 60%, 65%, 70%, 75%, 80%, 85%, 90%, 95%, 98%, 99% or 100% sequence identity with SEQ ID NO: 19. The amino acid sequence of the BBM2 protein can have at least about 50%, 55%, 60%, 65%, 70%, 75%, 80%, 85%, 90%, 95%, 98%, 99% or 100% sequence identity with SEQ ID NO: 20. In an embodiment, the BBM2 protein is encoded by a nucleotide sequence having at least about 50%, 55%, 60%, 65%, 70%, 75%, 80%, 85%, 90%, 95%, 98%, 99% or 100% sequence identity with SEQ ID NO: 21.

The amino acid sequence of the WOUND INDUCED DEDIFFERENTIATION 1 (WIND1) protein can have at least about 50%, 55%, 60%, 65%, 70%, 75%, 80%, 85%, 90%, 95%, 98%, 99% or 100% sequence identity with SEQ ID NO: 22. SEQ ID NO: 22 is the Arabidopsis thaliana WIND1 protein. In an embodiment, the WIND1 amino acid sequence is or is derived from AT1G78080, a homolog thereof, or a sequence having at least about 50%, 55%, 60%, 65%, 70%, 75%, 80%, 85%, 90%, 95%, 98%, 99% or 100% sequence identity with AT1G78080 or its homolog. In an embodiment, the WIND1 protein is encoded by a nucleotide sequence having at least about 50%, 55%, 60%, 65%, 70%, 75%, 80%, 85%, 90%, 95%, 98%, 99% or 100% sequence identity with SEQ ID NO: 23. The nucleotide sequence encoding the WIND1 protein can be, or can be derived from the gene AT1G78080, a homolog thereof or a sequence having at least about 50%, 55%, 60%, 65%, 70%, 75%, 80%, 85%, 90%, 95%, 98%, 99% or 100% sequence identity with AT1G78080 or its homolog. The percentage identity can be determined over the full length of the genomic sequence. Alternatively the percentage identity can be determined over the full length of the coding sequence of the gene.

Optionally, the morphogenic developmental polypeptide is a LEC1 (preferably any one of SEQ ID NO: 2, 8, 10, 12, 14, 16, 18, 20, or 22 in U.S. Pat. No. 6,825,397, which is disclosed herein by reference, or a homolog thereof having at least about 50%, 55%, 60%, 65%, 70%, 75%, 80%, 85%, 90%, 95%, 98%, 99% or 100% sequence identity thereto), a SHORT ROOT protein (SHR, preferably having SEQ ID NO: 30 or having a sequence encoded by SEQ ID NO: 31, or a homolog thereof having at least about 50%, 55%, 60%, 65%, 70%, 75%, 80%, 85%, 90%, 95%, 98%, 99% or 100% sequence identity thereto) or SCARECROW protein (SCR, preferably having SEQ ID NO: 32 or having a sequence encoded by SEQ ID NO: 33, or a homolog thereof having at least about 50%, 55%, 60%, 65%, 70%, 75%, 80%, 85%, 90%, 95%, 98%, 99% or 100% sequence identity thereto).

Preferably, the inducible promoter operably linked to the (first) sequence encoding the morphogenic developmental polypeptide, is further operably linked to a second or further morphogenic developmental polypeptide. In addition or alternatively, at least part of the cells of the plant tissue of step a) of the method provided herein comprises the (first) inducible promoter operably linked to the (first) sequence encoding the morphogenic developmental polypeptide and comprises a further inducible promoter linked to a second or further morphogenic developmental polypeptide. The first and further inducible promoter may be (substantially) the same or different inducible promoters. Transcription from the first and further inducible promoter may be activated upon binding the same trans-activator. Optionally, the first morphogenic developmental polypeptide and the second morphogenic developmental polypeptide are controlled by the same inducible promoter, or by promoters having (substantially) the same sequence. These elements (first inducible promoter operably linked to the first and optional further morphogenic developmental polypeptide(s), and/or the second inducible promoter operably linked to the optional further morphogenic developmental polypeptide(s)) may all be on the same construct. In addition or alternatively, the first and further morphogenic developmental polypeptide(s) may, each with their inducible promoter, be comprised in separate constructs.

The at least part of the cells comprising the sequence(s) encoding a regeneration factor(s) (i.e. morphogenic developmental polypeptide(s)) may further comprise one or more sequences encoding a trans-activator which, upon binding of the inducer, promotes or activates transcription of the one or more regeneration factor(s). In case of multiple sequences encoding a trans-activator are present in the plant cell(s), these sequences may be different and encode for different trans-activators preferably activated by different inducers. Each sequence encoding a trans-activator is preferably operably linked to an constitutively active promoter. Non-limiting examples of constitutive promoters include CaMV 35S, G10-90, CsV, TCTP2 and a UBQ10 promoter. Upon binding an inducer, the expressed trans-activator can bind to the inducible promoter that is operably linked to the at least one sequence encoding a regeneration factor and initiate transcription.

A trans-activator can be any suitable molecule that activates transcription upon binding an inducer. Preferably, the one or more trans-activators are trans-activators as described herein, preferably selected from the group consisting of GVG, XVE, ER-C1, GVECR, LhGR, TGV, AlcR, tetR-VP16 and tetR repressor protein. A preferred trans-activator is at least one of GVG, XVE of LhGR, preferably GVG.

The one or more trans-activators may be GVG, preferably encoded by a sequence having at least 50%, 60%, 70%, 75%, 80%, 85%, 90%, 95%, 96%, 97%, 98%, 99% or 100% sequence identity with any one of SEQ ID NO: 24. The one or more trans-activators may be XVE, preferably encoded by a sequence having at least 50%, 60%, 70%, 75%, 80%, 85%, 90%, 95%, 96%, 97%, 98%, 99% or 100% sequence identity with any one of SEQ ID NO: 25. The one or more trans-activators may be LhGR, preferably encoded by a sequence having at least 50%, 60%, 70%, 75%, 80%, 85%, 90%, 95%, 96%, 97%, 98%, 99% or 100% sequence identity with any one of SEQ ID NO: 26. The one or more trans-activators may be AlcR, preferably encoded by a sequence having at least 50%, 60%, 70%, 75%, 80%, 85%, 90%, 95%, 96%, 97%, 98%, 99% or 100% sequence identity with any one of SEQ ID NO: 26. Upon binding an inducer, a trans-activator may activate the inducible promoter linked to at least one sequence encoding the regeneration factor. As non-limiting examples, in case the trans-activator is GVG, the trans-activator may activate expression induced by an inducible promoter upon binding dexamethasone, or a derivative thereof; in case the trans-activator is LhGR, the trans-activator may activate expression induced by an inducible promoter upon binding dexamethasone, or a derivative thereof; and in case the trans-activator is XVE, the trans-activator may activate expression induced by an inducible promoter upon binding β-estradiol, or a derivative thereof. The skilled person readily understands that other combinations of a trans-activator—inducer—inducible promoter are equally suitable.

In a preferred embodiment, at least part of the plant cells of the plant tissue of step a) of the method provided herein comprises an expression construct comprising an inducible promoter operably linked to a sequence encoding at least one of a PLETHORA (PLT) polypeptide and a WUS/WOX homeobox polypeptide. Preferably, said construct comprises a first inducible promoter operably linked to a sequence encoding the PLETHORA (PLT) polypeptide and a further inducible promoter linked to the WUS/WOX homeobox polypeptide. The first and further inducible promoter may be the same or have (substantially) the same, or different inducible promoter sequences. Transcription from the first and further inducible promoter may be activated upon binding the same trans-activator. Optionally, the PLT polypeptide and the WUS/WOX polypeptide are controlled by the same inducible promoter. Hence, at least part of the plant cells of the plant tissue of step a) may comprise a first inducible promoter operably linked to a sequence encoding the PLETHORA (PLT) polypeptide and a second inducible promoter having (substantially) the same sequence as the first inducible promoter linked to a sequence encoding the WUS/WOX homeobox polypeptide. Preferably the PLT protein is PLT1 and the WUS/WOX homeobox polypeptide is WOX5.

Preferably, the sequence encoding the PLT protein and the sequence encoding the WUS/WOX homeobox polypeptide are each linked to a GVG-inducible promoter. Preferably, the sequence encoding the PLT1 protein and the sequence encoding the WOX5 homeobox polypeptide are each linked to a GVG-inducible promoter. Alternatively, the sequence encoding the PLT protein and the sequence encoding the WUS/WOX homeobox polypeptide are each linked to a XVE-inducible promoter. Preferably, the sequence encoding the PLT1 protein and the sequence encoding the WOX5 homeobox polypeptide are linked to a XVE-inducible promoter. Preferably, at least part of the plant cells of the plant tissue of step a) may comprise a first inducible promoter operably linked to a sequence encoding the PLETHORA (PLT) polypeptide and a second inducible promoter having (substantially) the same sequence as the first inducible promoter linked to a sequence encoding the WUS/WOX homeobox polypeptide, and a sequence encoding a transactivator that upon binding to an inducer is capable of activating the first inducible promoter. In case the first inducible promoter is an XVE-inducible promoter, said transactivator may be XVE that is induced by β-estradiol. In case the first inducible promoter is an GVG-inducible promoter, said transactivator may be GVG that is induced by dexamethasone.

In a preferred embodiment, the at least part of the cells of the plant tissue of step a) further comprises a second inducible promoter operably linked to a sequence encoding WIND1. The second inducible promoter comprises one or more elements for binding a (activated) second trans-activator. The element(s) binding a second trans-activator are preferably as defined herein. The element(s) are preferably linked to a minimal promoter (such as a minimal 35S promoter) or a CaMV 35S promoter. Optionally, the element(s) are linked to a tissue-specific promoter. Preferably, the sequence encoding the WIND1 protein is linked to an XVE-inducible promoter. Alternatively, preferably the sequence encoding the WIND1 protein is linked to an GVG-inducible promoter. Preferably, at least part of the plant cells of the plant tissue of step a) may comprise a GVG-inducible promoter operably linked to a sequence encoding a PLETHORA (PLT) polypeptide, GVG-inducible promoter linked to a sequence encoding a WUS/WOX homeobox polypeptide, an XVE-induced promoter operably linked to a sequence encoding a WIND1 protein, a sequence encoding XVE and a sequence encoding GVG. Preferably, the sequences encoding XVE and GVG are each operably linked to a promoter constitutive active in plant cells, preferably a CaMV 35S, G10-90, CsV, TCTP2 or a UBQ10 promoter. Preferably, the transactivators are operably linked to a CaMV 35S pormoter (SEQ ID NO: 28) and a CaMV terminator (SEQ ID NO: 29).

A preferred expression construct present in at least part of the cells of the plant tissue of step a) of the method provided herein is described in WO 2019/211296, which is incorporated herein in its entirety. Preferably the expression construct is the shoot regeneration vector (XVE-transactivated AmiRBR and AtWIND1; GVG-transactivated AtSHR, AtSCR, AtPLT1, AtPLT4, AtPLT5 and AtWOX5) or the shoot regeneration vector-2 (XVE-transactivated AtWIND1; GVG-transactivated AtPLT1 and AtWOX5) as described in example 1 and 2 of WO 2019/211296. Preferably, said expression construct is stably inserted in the plant cells.

Optionally, at least part of the plant cells of the plant tissue of step a) may, optionally in addition to the at least one sequence encoding a regeneration factor operably linked to an inducible promoter and/or the expression construct as defined herein, comprise a sequence encoding one or more components of a programmable nuclease for genome editing. Preferably said one or more components is a CRISPR endonuclease as defined herein and/or a TALEN.

In step b) of the method provided herein, the plant cell/plant tissue is exposed to a hydrogel. Preferably, the hydrogel is a hydrogel as defined herein above, i.e. a hydrogel comprising one or more compounds as defined herein, i.e. one or more inducers as defined herein.

Step b) may comprise a step of applying a hydrogel locally, for instance by applying a hydrogel bead onto a plant tissue. A bead may have a substantially spherical shape, and therefore the bead may be a sphere, but is not limited thereto. The bead may also have a non-spherical shape (a disc, a cube, a granule or the like).

The hydrogel bead is preferably a hydrogel bead as defined herein. In this embodiment, a compound may have a uniform distribution, or concentration, throughout the hydrogel bead. The gradient of the one or more compounds is formed when the compound diffuses out of the hydrogel and in the plant tissue. Preferably, the hydrogel bead comprises a limited concentration of one or more compounds, hence the concentration of the compound diffusing from the hydrogel is limited and preferably diminishes over time. The concentration of the one or more compounds diffusing from the hydrogel may be limited because of the concentration of the compound present in the hydrogel and/or because the hydrogel bead dehydrates and/or evaporates over time thereby diminishing or abolishing diffusion of the one or more compounds from the hydrogel.

The hydrogel, preferably the hydrogel bead, may comprise one or more inducers as a defined herein. A preferred compound is a an inducer of gene expression. Preferably the inducer of gene expression is selected from the group consisting of a steroid, an antibiotic, copper, ethanol, acetaldehyde and an insecticide. Preferably, the inducer is a steroid, preferably at least one of dexamethasone or a derivative thereof, or β-estradiol or a derivative thereof. Preferably, the hydrogel comprises one or more inducers for expressing the one or more regeneration factors comprised in at least part of the cells of the plant tissue of step a) of the method provided herein.

The method provided herein may further comprise a step c) of producing a regenerative plant structure, preferably a plant shoot. The shoot is preferably derived from a somatic embryonic structure. Optionally, a (somatic) embryogenic tissue is formed that does not itself form an embryo but is the site for secondary shoot organogenesis. Therefore, step c) is preferably performed under conditions suitable for formation of a regenerative plant structure, preferably conditions for shoot formation. These conditions are understood herein as at least being the minimal requirements of a plant cell to regenerate, which in general at least include normal growth conditions of said plant or plant cell. In a preferred embodiment the regeneration conditions require exposure of the plant tissue to a hydrogel as defined herein, wherein the hydrogel comprises one or more inducers, resulting in the expression of at least one of a PLT polypeptide and a WUS/WOX homeobox polypeptide in the plant tissue, preferably both a PLT polypeptide and a WUS/WOX homeobox polypeptide as defined herein, even more preferably, a PLT polypeptide, a WUS/WOX homeobox polypeptide and a WIND1 polypeptide as defined herein.

Optionally, step c) comprises the formation of callus prior to formation of a plant structure, preferably prior to shoot formation. Therefore, step c) may comprise the sub-steps of c1) allowing the plant tissue to form callus; and c2) allowing a plant structure, preferably a shoot and/or plant embryo, to grow from said callus, wherein optionally the culturing conditions of c1 and c2) are different. More in particular, step c1) may be performed under conditions suitable for the plant tissue to form callus; and step c2) may be performed under conditions suitable for the plant callus to form a plant structure, preferably a shoot. The skilled person is aware of conditions suitable for callus and/or regeneration. In a preferred embodiment step c1) and c2) require the exposure of the plant tissue to a hydrogel as defined herein, wherein the hydrogel comprises one or more inducers, resulting in the expression of at least one of a PLT polypeptide and a WUS/WOX homeobox polypeptide in the plant tissue.

Optionally, the plant structure, preferably a shoot, is produced without a required exposure to plant growth hormones, i.e. “Hormone-independent shoot regeneration”. Preferably a regenerative plant structure, preferably a plant embryo and/or a plant shoot, is formed by contacting the plant tissue locally to a hydrogel, preferably placing a hydrogel bead comprising the one or more inducers onto a plant tissue as defined herein, thereby producing a gradient of one or more inducers in said plant tissue.

The plant structure, preferably the plant shoot, may be formed by contacting a plant tissue comprising an expression construct as defined herein to a gradient of one or more inducers, wherein the gradient is formed by placing the hydrogel bead onto the plant tissue. The inventors discovered that by placing a hydrogel bead with one or more inducers on a plant tissue of a plant comprising a construct as defined herein, embryonic structures are formed at a certain distance from the hydrogel bead that effectively grow out to full plant structures via shoot formation from which full plants can be grown, as opposed to plant tissue placed on a medium comprising a predetermined concentration of one or more inducers thereby contacting one full surface of said plant tissue with said medium. In the latter circumstance, many morphogenic structures were formed on the plant tissue, however, all degenerated and failed to develop to full plant structures.

The method may further comprise a step of generating a plant from the produced plant structure, preferably from the produced plant shoot. Preferably said plant comprises at least one inflorescence and/or is capable of vegetative propagation. Optionally the method may further comprise a step of producing seed and/or a progeny plant of the generated plant by vegetative, sexual or apomictic reproduction.

In a further aspect, provided is a plant obtainable from the method provided herein. Hence the plant may be a transgenic plant and/or mutant plant, e.g. comprising at least one regeneration factor and/or trans-activator as defined herein (preferably stably inserted in its genome), a transgene and optionally a mutation in a sequence of interest. The plant may be a man-made plant. The plant may be obtainable using targeted genome editing such as CRISPR technology as described herein or TALEN. Optionally, said plant may comprise a sequence encoding one or more components of the CRISRP complex, i.e. a sequence encoding a CRISPR endonuclease and/or a sequence encoding one or more RNAs for guiding said CRISPR endonuclease (a crRNA, a tracrRNA and/or a single guide RNA comprising or consisting of a crRNA linked to a tracrRNA). Optionally, the sequence encoding the one or more components of the CRISPR-complex is removed, e.g. by crossing and selection, after inducing the mutation in the sequence of interest. Optionally, the plant obtainable by the method provided herein is a plant that differs from the naturally occurring plant only by having a mutation in a sequence of interest. Preferably, the transgene or mutation in the sequence of interest is located in germline or germline progenitor cells and/or tissue and/or plant part for clonal propagation. Preferably, the plant provided herein is not, or is not exclusively, obtained by an essentially biological process. The plant provided herein preferably differs at least from a plant occurring in nature, in that it contains at least one regeneration factor and/or trans-activator as defined herein, a transgene, and a optionally mutation in a sequence of interest. The transgene or mutation in the sequence of interest is preferably located in at least the germline or germline progenitor cells and/or tissue and/or plant part for clonal propagation of the plant. The transgene or mutation in the sequence of interest is preferably located in at least the L2-shoot meristem layer. The transgene or mutation in the sequence of interest is preferably present in at least one of the pollen and egg of the plant. Optionally, the transgene or construct comprising at least one regeneration factor and/or trans-activator regeneration construct is outcrossed in subsequent generations.

Further provided is offspring or seed from the plant produced by the method provided herein. The offspring may be produced by sexual or α-sexual (vegetative) propagation. The offspring preferably comprises at least one regeneration factor and/or trans-activator as defined herein, a transgene or mutation in a sequence of interest as defined herein.

Also provided is a plant part or plant product derived from a plant obtained from the method provided herein. Optionally, said plant part or plant product is characterized in that it comprises at least one regeneration factor and/or trans-activator as defined herein. Optionally, the plant part or plant product is characterized in that it comprises a transgene or mutation in a sequence of interest. Such genetic material may be genomic DNA or fragments of genomic DNA. Such genetic material may be mitochondrial DNA or fragments of mitochondrial DNA. Such hereditary material may be chloroplast DNA or fragments of chloroplast DNA. The plant part may be propagating or non-propagating material.

Also provided is a combination of a plant cell and a hydrogel. The plant cell is preferably comprised in a plant tissue, preferably a plant tissue as defined herein. Alternatively or in addition, the hydrogel is a hydrogel as defined herein. Preferably the hydrogel is a hydrogel bead comprising one or more inducers as defined herein. The hydrogel, preferably a hydrogel bead, is preferably for a non-medical use. Preferably, the hydrogel, preferably the hydrogel bead, is not used for a medical treatment of an animal, preferably is not used for the medical treatment of a mammal, preferably not used for the medical treatment of a human. The hydrogel bead can be unsuitable for medical treatment of an animal, mammal and/or human. Optionally the hydrogel bead is for use in or on plants. Preferably, the hydrogel or hydrogel bead further comprises one or more compounds that are specific for plants, for instance one or more plant specific bioactive compounds such as, but not limited to, plant hormones, programmable nucleases (or nucleic acid encoding the same) designed to target genomic plant sequences, vectors designed for expressing proteins in plant cells (e.g. encoding plant proteins and/or plant-specific promoter sequences), mRNA encoding plant proteins or siRNA or miRNA for targeting plant transcripts. Preferably, the hydrogel or hydrogel bead is applied topically on (or contacted with the exterior of) a plant part, plant structure, plant tissue, plant organ, plant organ system or whole plant. Preferably, the hydrogel or hydrogel bead is applied topically on (or contacted with the exterior of) a plant leaf, a plant shoot or a plant callus. Further provided is the use of a hydrogel, preferably a hydrogel bead, as defined herein for inducing regeneration of a plant tissue
All patent and literature references cited in the present specification are hereby incorporated by reference in their entirety.

### Examples

### Example 1. Hydrogel-Carriers for Induction of Regeneration in S. Lycopersicum

Plant materials: A stable transgenic single insertion tomato line was generated by transformation of a tomato cells (Moneymaker, The Netherlands) with the construct pKG11052, as described previously (Example 2 of WO 2019/211296). The construct pKG11052 comprises the following promoter—transgene expression cassettes:
CaMV 35S—XVE
CaMV 35S—GVG
XVE inducible promoter—WIND1
GVG inducible promoter—PLT1
GVG inducible promoter—WOX5
CaMV 35S—erGFP
   Seeds were sterilized (70% ethanol rinse for 5 minutes, a 15 minutes wash with 1% bleach followed by 4 times rinsing with sterile tap water) and germinated on moist sterile Whatman filter paper (100 mm; Tisch Products, product code: 10311810. Germinated seeds were transferred to sterile containers containing 100 ml of solid Murashige & Skoog medium including vitamins, 20 g/l sucrose, 0.8% micro-agarose and 0.5 g/l MES (pH5.8).
   Cotyledon and young leaf segments were excised from 1 week old seedlings and positioned on a solid Murashige & Skoog medium including vitamins, 20 g/l sucrose, 0.8% micro-agarose and 0.5 g/l MES (pH5.8).
   Generation of inductive hydrogel beads: Hydrogel beads were generated by pipetting 10 μl of an 1.6% alginate solution in an excess (over 20 ml) of 1% CaCl₂ solution. Beads were harvested and equilibrated in liquid Murashige & Skoog including vitamins, 20 g/l sucrose, 0.5 g/l MES (pH5.8) for over an hour. Beads were subsequently equilibrated in an inducer solution containing liquid Murashige & Skoog including vitamins medium supplemented with 20 g/l sucrose, 0.5 g/l MES (pH5.8), 10 μM Dexamethason (50 mM stock solution in DMSO, diluted 5000 times in final solution) and 1 μM Estradiol (10 mM stock solution in DMSO, diluted 10000 times in final solution) to create inductive beads.

### Results

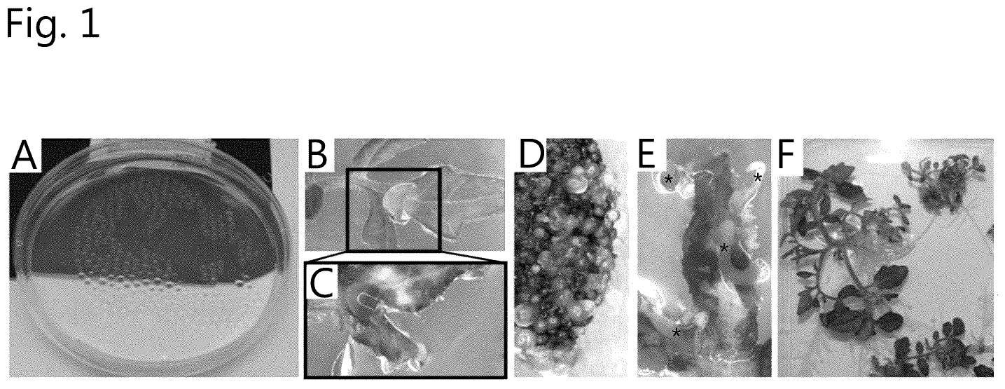

Inductive hydrogel carriers were generated as described above (FIG. 1A.) and placed on the tissue surfaces of cotylodon and young leaf tissue segments using a fine tweezer (FIG. 1 B.). Local morphogenic events with visually observable organs were present within two and a half week after inductive hydrogel bead application (FIG. 1C). In contrast, the conventional method (inducer supplementation to the growth medium) repeatedly yielded massive morphogenic events throughout the tissue surface. The formed morphogenic structures undergo developmental arrest (FIG. 1D). In contrast, morphogenic events from local hydrogel bead mediated induction resulted in the development of embryonic structures and viable seedlings (FIG. 1E). Subsequently, regenerant shoots were developed from the induced embryogenic structures (FIG. 1F).
Example 2. Hydrogel-Carriers for Induction of Regeneration in C. annuum c.v. Maor.
Plant materials: A stable transgenic Maor pepper (Capsicum Annuum c.v. Maor; Israel) was generated by transformation of Maor pepper cells with the construct pKG11052 (described above, see example 1). Seeds were sterilized (70% ethanol rinse for 5 minutes, a 15 minutes wash with 1% bleach followed by 4 times rinsing with sterile tap water) and germinated on solid Murashige & Skoog medium including vitamins and supplemented with 20 g/l sucrose, 0.8% of micro-agarose and 0.5 g/l MES (pH5.8). Cotyledons were dissected of from 10 day old seedlings and cut twice to obtain three explants. Explants were pre-cultured for 2 days on co-cultivation medium (CCM) (Heidmann et al, 2011 Plant Cell Rep. 30 (6): 1107-15) supplemented with 40 mg/l Acetosyringone, 1.6% (w/v) glucose, 0.7% micro-agarose, 2 mg/l zeatin riboside and 0.1 mg/l indole-3-acetic acid. Explants were submerged for 30 minutes in liquid CCM containing a suspension of Agrobacterium tumefaciens strain GV3101 (OD600=0.3) carrying pKG11052 and 40 mg/l acetosyringone. Explants were blotted dry and co-cultivated for two days under dim light on co-cultivation medium supplemented with 40 mg/l Acetosyringone, 1.6% (w/v) glucose, 0.7% micro-agarose, 2 mg/l zeatin riboside and 0.1 mg/l indole-3-acetic acid, wherein the light was dimmed by placing cellulose chromatography paper (GE Whatman, grade: 3 mm CHR, Cat no: 3030-931) on top of the plates. After co-cultivation, explants were cultivated at 23° C. and full light conditions (3000 lux, 16/8 h photoperiod) on co-cultivation medium supplemented with 1 mg/l thidiazuron, 100 mg/l kanamycin sulfate and 500 mg/l cefotaxime. The explants were subcultured every month onto fresh medium. GFP expressing micro-calli were isolated from the tissue explants and subcultured until showing greening and first signs of production of leaf-like structures (3˜4 months after transformation). Calli with incipient leaf-like structures were further subcultured on elongation medium (Heidmann et al, supra) supplemented with 1.6% (w/v) glucose, 1 mg/l inositol, 20 mg/l adenine sulfate, 200 mg/l casein hydrolysate, 1 mg/l gibberellic acid 3, 4 mg/l benzylaminopurine and 30 μM silverthiosulfate. Once elongated, leaf-like structures were dissected of the callus and cultivated on solid Murashige & Skoog medium including vitamins and supplemented with 20 g/l sucrose, 0.8% micro-agarose and 0.5 g/l MES (pH5.8). Freshly dissected leaf-like structures were used for induction assays.
Generation of inductive hydrogel beads: Hydrogel beads were generated by pipetting 10 μl of an 1.6% alginate solution in an excess (over 20 ml) of 1% CaCl₂ solution. Beads were harvested and equilibrated in liquid Murashige & Skoog incl. vitamins medium containing sucrose (20 g/l), 0.5 g/l MES (pH5.8) for over an hour. Beads were subsequently equilibrated in an inducer solution containing liquid Murashige & Skoog incl. vitamins medium containing sucrose (20 g/l), 0.5 g/l MES (pH5.8), 10 uM Dexamethason and 1 uM Estradiol) to create inductive beads.

### Results

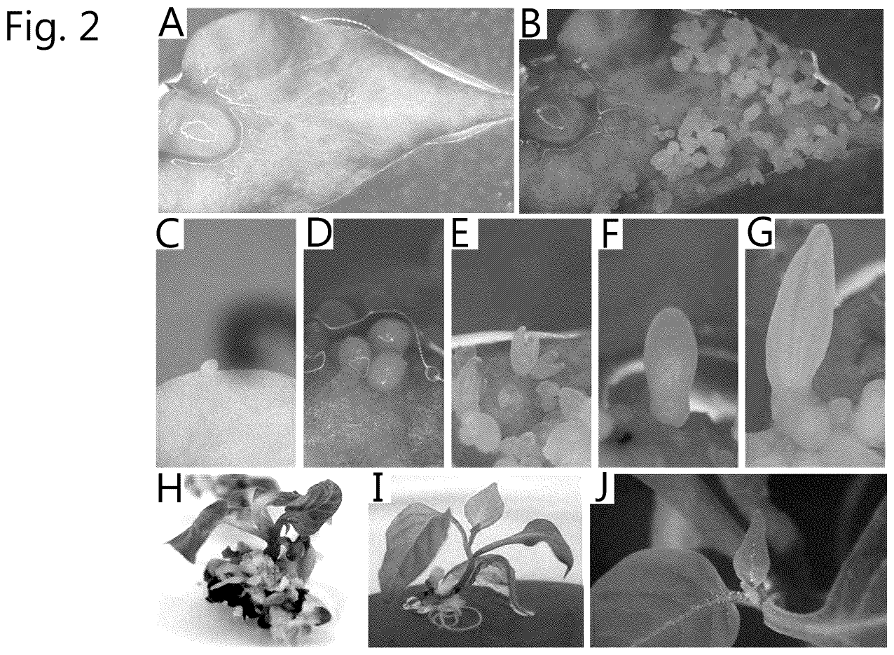

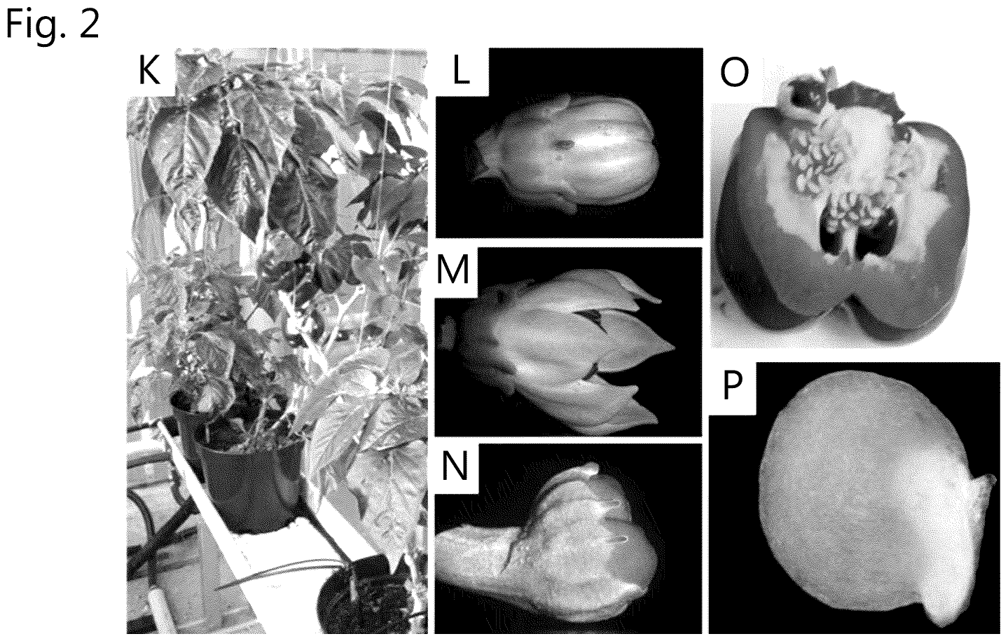

Maor pepper (Capsicum annuum) is known for being a recalcitrant plant, showing no regeneration with any conventional tissue culture regeneration procedure. Inductive hydrogels were generated and placed on the surfaces of freshly dissected leaf-like structure tissue segments with a fine tweezer (FIG. 2A.). Local morphogenic events were induced with visually observable somatic embryos present within 19 days after inductive hydrogel beads application (FIG. 2B). The morphology of the somatic embryos was well defined with all subsequent stages of embryo development present within the population (FIG. 2C-G). Embryos germinated while remaining attached to the callus (FIG. 2H). Regenerant plants also developed from isolated mature embryos (dissected of the callus) cultivated on solid Murashige & Skoog medium including vitamins, 20 g/l sucrose, 2 mg/l GA3, 0.8% micro-agarose and 0.5 g/l MES (pH5.8) (FIG. 2I,J). The plantlet was further grown into a regenerant plant carrying mature transgenic fruits (FIG. 2K). FIGS. 2L-2N depict the subsequent stages of flower development during fruit set. More in particular FIG. 2L shows a closed flower bud, FIG. 2M an open flower bud and FIG. 2N a fertilized flower bud. FIG. 2O shows the mature transgenic fruit having transgenic seed set and FIG. 2P shows that there is clear GFP expression in the Maor seed.
Hence exposure of the plant cells to inductive hydrogel beads results in plant regeneration. In addition, the transgene (GFP) remains in the germplasm of the regenerated plant and the transgene is thus carried over to the progeny plants.

1. A method for producing a plant shoot, comprising the steps of:
a) providing a plant tissue comprising a sequence encoding at least one regeneration factor under the control of an inducible promoter;
b) locally contacting the plant tissue to an inductive hydrogel bead comprising an inducer, wherein the inducer is a compound capable of inducing the inducible promoter, optionally upon binding to a trans-activator comprised in the plant tissue; and
c) allowing the shoot to be produced from the plant tissue.

2. A method for producing a plant shoot according to claim 1, wherein the hydrogel is a hydrogel bead, and wherein the bead is a macrosphere having a diameter of about 0.1-10 millimetre (mm), or a microsphere having a diameter of about 0.1-100 micrometre (μm).

3. A method for producing a plant shoot according to claim 1, wherein the inducer is a steroid,

4. A method for producing a plant shoot according to claim 3, wherein the steroid is at least one of dexamethasone or a derivative thereof and wherein the steroid is β-estradiol or a derivative thereof.

5. A method according to claim 1,
wherein the inducer is dexamethasone or a derivative thereof, and wherein the plant tissue of step a) comprises trans-activator that is a GVG protein; and/or,
wherein the inducer is β-estradiol or a derivative thereof, and wherein the plant tissue of step a) comprises trans-activator that is a XVE protein.

6. A method according to claim 1, wherein the concentration of the inducer in the hydrogel bead is about 1 nM-100 μM.

7. A method according to claim 1, wherein the at least one regeneration factor comprises at least one of a PLETHORA (PLT) polypeptide and a WUS/WOX homeobox polypeptide, wherein preferably:
i) the PLT polypeptide is selected from the group consisting of PLT1, PLT2, PLT3, PLT4, PLT5 and PLT7, preferably PLT1; and
ii) the WUS/WOX homeobox polypeptide is selected from the group consisting of WUS1, WUS2, WUS3, WOX2A, WOX4, WOX5, or WOX9, preferably WOX5.

8. A method according to claim 7, wherein the at least one regeneration factor comprises PLT1 and WOX5, wherein the PLT1- and WOX5-encoding sequences are each operably linked to an inducible promoter, preferably a GVG-inducible promoter.

9. A method according to claim 1, wherein the at least one regeneration factor comprises WIND1, and wherein the WIND1-encoding sequence is operably linked to an inducible promoter, preferably XVE-inducible promoter.

10. A method according to claim 1, wherein the hydrogel is an alginate or a derivative thereof.

11. A method according to claim 1, wherein the plant tissue in step a) comprises an expression construct encoding the at least one regeneration factor under the control of an inducible promoter, and optionally encoding a trans-activator.

12. A method according to claim 1, wherein the plant tissue of step a) is a hypocotyl or a leaf, wherein the pant tissue is preferably any one of a cotyledon, a first true leaf, and a young (axillary) leaf.

13. A method according to claim 1, further comprising a step of regenerating a plant from the produced plant shoot.

14. A combination of a hydrogel bead comprising an inducer of gene expression as defined in claim 1 and a plant cell.

15. (canceled)


SEQUENCE LISTING (35 sequences)

SEQ ID NO: 1 - DNA, 186 bp, synthetic construct
  misc_feature 1..186: note = UAS
  source 1..186: mol_type = other DNA
ggagtgaaggatcgggtgacagccctccgagcgggtgacagccctccgaagcgggtgacagccctccgacgtcgggtgacagccctccgagcgggtgacagccctccgagcgggtgacagccctccgctgcagcaagacccttcctctatataaggaagttcatttcatttggagaggacagccca

SEQ ID NO: 2 - DNA, 318 bp, synthetic construct
  misc_feature 1..318: note = LacOp
  source 1..318: mol_type = other DNA
aaagaagaaagggaagagaaagaattgtgagcgctcacaattgaaagactagaaagaagaaagggaagagaaagaattgtgagcgctcacaattgaaagactagaaagaagaaagggaagagaaagaattgtgagcgctcacaattgaaagactagaaagaagaaagggaagagaaagaattgtgagcgctcacaattgaaagactagaaagaagaaagggaagagaaagaattgtgagcgctcacaattgaaagactagaaagaagaaagggaagagaaagaattgtgagcgctcacaattgaaagactagtggatc

SEQ ID NO: 3 - DNA, 355 bp, synthetic construct
  misc_feature 1..355: note = LexAop
  source 1..355: mol_type = other DNA
ggagagcttgggctgcaggtcgaggctaaaaaactaatcgcattatcatcccctcgacgtactgtacatataaccactggttttatatacagcagtactgtacatataaccactggttttatatacagcagtcgacgtactgtacatataaccactggttttatatacagcagtactgtacatataaccactggttttatatacagcagtcgaggtaagattagatatggatatgtatatggatatgtatatggtggtaatgccatgtaatatgctcgactctaggatcttcgcaagacccttcctctatataaggaagttcatttcatttggagaggacacgctgaagctagtc

SEQ ID NO: 4 - protein, 182 aa, Arabidopsis thaliana
MSFSVKGRSLRGNNNGGTGTKCGRWNPTVEQLKILTDLFRAGLRTPTTDQIQKISTELSFYGKIESKNVFYWFQNHKARERQKRRKISIDFDHHHHQPSTRDVFEISEEDCQEEEKVIETLQLFPVNSFEDSNSKVDKMRARGNNQYREYIRETTTTSFSPYSSCGAEMEHPPPLDLRLSFL

SEQ ID NO: 5 - DNA, 549 bp, Arabidopsis thaliana
  source 1..549: mol_type = other DNA
atgtctttctccgtgaaaggtcgaagcttacgtggcaacaataacggaggaacggggacgaagtgcgggagatggaatccaacggtggagcagttgaagatattgactgatctgtttcgagccggtcttagaactccaacaactgatcagattcagaagatctctacggagctcagtttctacggcaagatagagagcaagaatgttttctattggtttcagaatcataaggctagggagaggcagaaacgtcgtaaaatctccattgattttgatcatcatcatcatcaaccatcaactagagatgtttttgaaataagcgaagaagattgtcaagaggaagagaaggtgatagagacattacaactctttccggtgaattcatttgaagactccaactccaaggtggacaaaatgagagctagaggcaataaccagtaccgtgaatatattcgagagaccaccacgacgtcgttttctccatactcatcatgtggagctgaaatggaacatccaccgccattagatcttcgattaagctttctttaa

SEQ ID NO: 6 - protein, 574 aa, Arabidopsis thaliana
MNSNNWLGFPLSPNNSSLPPHEYNLGLVSDHMDNPFQTQEWNMINPHGGGGDEGGEVPKVADFLGVSKPDENQSNHLVAYNDSDYYFHTNSLMPSVQSNDVVVAACDSNTPNNSSYHELQESAHNLQSLTLSMGTTAGNNVVDKASPSETTGDNASGGALAVVETATPRRALDTFGQRTSIYRGVTRHRWTGRYEAHLWDNSCRREGQSRKGRQVYLGGYDKEDKAARSYDLAALKYWGPSTTTNFPITNYEKEVEEMKHMTRQEFVAAIRRKSSGFSRGASMYRGVTRHHQHGRWQARIGRVAGNKDLYLGTFSTEEEAAEAYDIAAIKFRGLNAVTNFEINRYDVKAILESSTLPIGGGAAKRLKEAQALESSRKREAEMIALGSSFQYGGGSSTGSGSTSSRLQLQPYPLSIQQPLEPFLSLQNNDISHYNNNNAHDSSSFNHHSYIQTQLHLHQQTNNYLQQQSSQNSQQLYNAYLHSNPALLHGLVSTSIVDNNNNNGGSSGSYNTAAFLGNHGIGIGSSSTVGSTEEFPTVKTDYDMPSSDGTGGYSGWTSESVQGSNPGGVFTMWNE

SEQ ID NO: 7 - DNA, 1725 bp, Arabidopsis thaliana
  source 1..1725: mol_type = other DNA
atgaattctaacaactggcttggctttcctctttcaccgaacaactcttctttgcctcctcatgaatacaaccttggcttggtcagcgaccatatggacaacccttttcaaacacaagagtggaatatgatcaatccacacggtggaggaggagatgaaggaggagaggttccaaaagtggccgattttctcggtgtgagcaaaccggacgaaaaccaatccaaccacctagtagcttacaacgactcagactactacttccataccaatagcttgatgcctagcgtccaatcaaacgatgtcgttgtagcagcttgtgactccaatactcctaacaacagtagctatcatgagcttcaagagagtgctcacaatctacagtcacttactttgtccatggggaccaccgctggtaataatgttgtagacaaagcttcaccatccgagaccaccggggataacgctagcggtggagcactagccgttgttgagacggccacgccaagacgtgcattggacactttcggacaacgaacctcgatctatcgtggtgtcacaagacatcgatggactggtcgatatgaggctcatctatgggataatagttgtagaagggaaggccagtctaggaaaggaagacaagtttacttgggtggatatgacaaagaagataaagcagcaagatcatatgatctagctgcacttaagtactggggtccttcaactactactaatttccccattacaaactacgagaaagaagtagaggaaatgaagcacatgacgagacaagagttcgtggctgccattagaaggaaaagtagtggattttcgagaggcgcttcgatgtatcgaggagttacaaggcatcaccaacatggaagatggcaagcaaggatcggccgagtcgccggaaacaaagacctctacttgggaacttttagcactgaggaagaagcagcagaagcttacgatatagctgcaataaagtttagaggacttaatgcagtgaccaacttcgagatcaaccggtacgacgtgaaagccattctagagagtagcactcttcccatcggaggaggcgcagctaaacggctcaaagaagctcaagctcttgagtcttcaaggaaacgcgaggcggagatgatagcccttggttcaagtttccagtacggtggtggctcgagcacaggctctggctccacctcatcaagacttcagcttcaaccttaccctctaagcattcaacaaccattagagccttttctatctcttcagaacaatgacatctctcattacaacaacaacaatgctcacgattcctcctcttttaatcaccatagctatatccagacacaacttcatctccaccaacagaccaacaattacttgcagcaacagtcgagccagaactctcagcagctctacaatgcgtatcttcatagcaatccggctctgcttcatggacttgtctctacctctatcgttgacaacaataataacaatggaggctctagtgggagctacaacactgcagcatttcttgggaaccacggtattggtattgggtccagctcgactgttggatcgaccgaggagtttccaaccgttaaaacagattacgatatgccttccagtgatggaaccggagggtatagtggttggaccagtgagtctgttcaggggtcaaaccctggtggtgttttcactatgtggaatgagtaa

SEQ ID NO: 8 - protein, 568 aa, Arabidopsis thaliana
MNSNNWLAFPLSPTHSSLPPHIHSSQNSHFNLGLVNDNIDNPFQNQGWNMINPHGGGGEGGEVPKVADFLGVSKSGDHHTDHNLVPYNDIHQTNASDYYFQTNSLLPTVVTCASNAPNNYELQESAHNLQSLTLSMGSTGAAAAEVATVKASPAETSADNSSSTTNTSGGAIVEATPRRTLETFGQRTSIYRGVTRHRWTGRYEAHLWDNSCRREGQSRKGRQVYLGGYDKEEKAARAYDLAALKYWGPSTTTNFPITNYEKEVEEMKNMTRQEFVASIRRKSSGFSRGASMYRGVTRHHQHGRWQARIGRVAGNKDLYLGTFSTEEEAAEAYDIAAIKFRGLNAVTNFEINRYDVKAILESNTLPIGGGAAKRLKEAQALESSRKREEMIALGSNFHQYGAASGSSSVASSSRLQLQPYPLSIQQPFEHLHHHQPLLTLQNNNDISQYHDSFSYIQTQLHLHQQQTNNYLQSSSHTSQLYNAYLQSNPGLLHGFVSDNNNTSGFLGNNGIGIGSSSTVGSSAEEEFPAVKVDYDMPPSGGATGYGGWNSGESAQGSNPGGVFTMWNE

SEQ ID NO: 9 - DNA, 1707 bp, Arabidopsis thaliana
  source 1..1707: mol_type = other DNA
atgaattctaacaactggctcgcgttccctctatcaccaactcactcttctttgccgcctcacattcactcttcacaaaattctcatttcaatctaggtttggtcaacgacaatatcgacaacccttttcaaaaccaaggatggaatatgatcaatccacatggtggaggcggcgaaggtggagaggttccaaaagtggctgatttcttaggagtgagcaaatcgggggatcatcacaccgatcacaacctcgtaccttataacgacattcatcaaaccaacgcctccgactactactttcaaaccaatagcttgttacctacagtcgtcacttgtgcctctaatgctcctaataattatgagcttcaagagagtgcacacaatttgcaatctctcactctctctatgggaagtactggagctgccgctgcagaagtcgccactgtgaaagcctcgccggctgagactagtgccgataatagtagcagcactaccaacacaagtggaggagccatcgttgaggctacaccgagacggactttggaaacttttggacaacgaacctctatctatcgtggagttacaagacatagatggaccggtagatatgaagctcatctttgggataatagctgtagaagagaaggacaatcaaggaaaggaagacaagtctacttaggtgggtatgacaaagaagagaaagcagccagagcatatgatctagctgcacttaaatattggggtccctctactactaccaactttccgataactaactacgagaaggaagtagaggagatgaaaaacatgacgagacaagagtttgtggcttctataagaaggaaaagtagcggattctcgcgtggtgcatccatgtatcgtggagtaacaaggcatcatcaacatggaagatggcaagcaaggatcggccgagttgctggaaacaaagatctctacttgggaacattcagcacggaggaagaagcagcagaagcttatgacatagctgcgataaagtttcgaggtctaaacgcggttacaaactttgagataaatcggtatgatgtgaaagccatcctggagagcaacacacttcctataggaggtggtgcggctaaacggctcaaagaagctcaagctctagaatcatcaagaaaacgagaggaaatgatagccctcggatcaaatttccatcaatatggtgcagcgagcggctcgagctctgttgcttccagctctaggcttcagcttcaaccttaccctctaagcattcaacaaccttttgagcatcttcatcatcatcagcctttacttactctacagaacaacaacgatatctctcagtatcatgattcctttagttacattcagacgcagcttcatcttcaccaacaacaaaccaacaattacttgcagtcttctagtcacacttcacagctctacaatgcttatcttcagagtaaccctggtctgcttcatggatttgtctctgataataacaacacttcagggtttcttggaaacaatgggattggtattgggtcaagctctaccgttggatcatcggctgaggaagagtttccagccgtgaaagtcgattacgatatgcctccttccggtggagctacagggtatggaggatggaatagtggagagtctgctcaaggatcgaatccaggaggtgttttcacgatgtggaatgaataa

SEQ ID NO: 10 - protein, 604 aa, Arabidopsis thaliana
MMAPMTNWLTFSLSPMEMLRSSDQSQFVSYDASSAASSSPYLLDNFYGWSNQKPQEFFKEEAQLAAAASMADSTILTTFVDPQSHHSQNHIPKLEDFLGDSSSIVRYSDNSQTDTQDSSLTQIYDPRHHHNQTGFYSDHHDFKTMAGFQSAFSTNSGSEVDDSASIGRTHLAGDYLGHVVESSGPELGFHGGSTGALSLGVNVNNNTNHRNDNDNHYRGNNNGERINNNNNNDNEKTDSEKEKAVVAVETSDCSNKKIADTFGQRTSIYRGVTRHRWTGRYEAHLWDNSCRREGQARKGRQVFYSFFGMCYLIWGCILALLKINSGYDKEDKAARAYDLAALKYWNATATTNFPITNYSKEVEEMKHMTKQEFIASLRRKSSGFSRGASIYRGVTRHHQQGRWQARIGRVAGNKDLYLGTFATEEEAAEAYDIAAIKFRGINAVTNFEMNRYDVEAIMKSALPIGGAAKRLKLSLEAAASSEQKPILGHHQLHHFQQQQQQQQLQLQSSPNHSSINFALCPNSAVQSQQIIPCGIPFEAAALYHHHQQQQQHQQQQQQQNFFQHFPANAASDSTGSNNNSNVQGTMGLMAPNPAEFFLWPNQSY

SEQ ID NO: 11 - DNA, 1815 bp, Arabidopsis thaliana
  source 1..1815: mol_type = other DNA
atgatggctccgatgacgaactggttaacgttttctctgtcaccaatggagatgttgaggtcatctgatcagtctcagtttgtctcctatgacgcttcttccgccgcttcctcctctccttatctcctcgataatttctatggttggtcaaaccaaaaacctcaggagtttttcaaagaagaagctcagttagcagcagcagcttcaatggcggattcaacaatcttaacaacattcgtagacccacaatctcatcattctcagaatcacatcccaaagctcgaagattttctcggtgactcttcttctatcgttcgttactccgacaacagtcaaaccgacacacaagactcttccctcactcaaatctacgatccacgtcaccaccataaccaaaccggcttttactccgatcaccacgatttcaaaaccatggccggttttcaatccgctttctctactaactccggttcagaggtcgatgactctgcttctatcggaaggactcatcttgctggagactatttgggacacgtggttgaatcttctggtccggagctagggtttcacggtggatctaccggagctttgtcacttggtgttaacgtcaataacaatactaatcaccggaatgataatgataatcattaccgaggcaataacaatggtgagagaatcaacaacaacaacaacaatgacaacgagaagacagattctgagaaggagaaggctgttgtggctgtggaaacatcagattgttctaataagaagattgctgatacgtttggtcaaaggacttcgatttacagaggtgttactcgacatagatggacgggaagatatgaagcacatctatgggataatagctgtaggagagaaggtcaggccaggaagggacgtcaagtcttctactctttttttggcatgtgctacttgatatggggatgcattttggctctgcttaaaatcaacagtggatatgacaaagaagataaggcagctcgagcttacgatttagcagctctgaaatactggaatgctactgctaccaccaatttccctattacgaattactcgaaagaagtggaggaaatgaagcacatgaccaagcaagagttcattgcctccctcaggaggaagagtagcggtttctctagaggagcttcgatataccgaggtgttacaaggcatcatcaacaaggacgttggcaagcaaggattggccgagttgctgggaacaaagatctttaccttggaacctttgcaacggaagaggaagcagctgaagcgtatgacatagcagcaatcaaattcagaggaataaacgctgtaactaactttgagatgaaccgttacgacgttgaagccatcatgaagagtgcacttcccatcggtggtgcagctaaacgtcttaagctctctttggaagctgctgcttcatcagagcagaaaccaatcctcggtcatcatcaacttcaccatttccagcaacaacaacaacaacaacagcttcagcttcagtcatctcctaatcacagtagcattaacttcgctctctgtcctaattcagcagttcagtctcaacagatcattccttgtggaatcccttttgaagcagctgctctttaccaccaccaccaacaacaacagcaacaccaacagcagcagcagcaacagaacttcttccagcattttccggcgaatgcagcttctgactcgaccgggtctaacaacaactccaacgttcagggaacaatgggacttatggcaccaaatccggctgagttcttcctctggcctaatcagtcttactaa

SEQ ID NO: 12 - protein, 584 aa, Arabidopsis thaliana
MNSMNNWLGFSLSPHDQNHHRTDVDSSTTRTAVDVAGGYCFDLAAPSDESSAVQTSFLSPFGVTLEAFTRDNNSHSRDWDINGGACNNINNNEQNGPKLENFLGRTTTIYNTNETVVDGNGDCGGGDGGGGGSLGLSMIKTWLSNHSVANANHQDNGNGARGLSLSMNSSTSDSNNYNNNDDVVQEKTIVDVVETTPKKTIESFGQRTSIYRGVTRHRWTGRYEAHLWDNSCKREGQTRKGRQVYLGGYDKEEKAARAYDLAALKYWGTTTTTNFPLSEYEKEVEEMKHMTRQEYVASLRRKSSGFSRGASIYRGVTRHHQHGRWQARIGRVAGNKDLYLGTFGTQEEAAEAYDIAAIKFRGLSAVTNFDMNRYNVKAILESPSLPIGSSAKRLKDVNNPVPAMMISNNVSESANNVSGWQNTAFQHHQGMDLSLLQQQQERYVGYYNGGNLSTESTRVCFKQEEEQQHFLRNSPSHMTNVDHHSSTSDDSVTVCGNVVSYGGYQGFAIPVGTSVNYDPFTAAEIAYNARNHYYYAQHQQQQQIQQSPGGDFPVAISNNHSSNMYFHGEGGGEGAPTFSVWNDT

SEQ ID NO: 13 - DNA, 1755 bp, Arabidopsis thaliana
  source 1..1755: mol_type = other DNA
atgaactcgatgaataactggttaggcttctctctctctcctcatgatcaaaatcatcaccgtacggatgttgactcctccaccaccagaaccgccgtagatgttgccggagggtactgttttgatctggccgctccctccgatgaatcttctgccgttcaaacatcttttctttctcctttcggtgtcaccctcgaagctttcaccagagacaataatagtcactcccgagattgggacatcaatggtggtgcatgcaataacattaacaataacgaacaaaatggaccaaagcttgagaatttcctcggccgcaccaccacgatttacaataccaacgagaccgttgtagatggaaatggcgattgtggaggaggagacggtggtggtggcggctcactaggcctttcgatgataaaaacatggctgagtaatcattcggttgctaatgctaatcatcaagacaatggtaacggtgcacgaggcttgtccctctctatgaattcatctactagtgatagcaacaactacaacaacaatgatgatgtcgtccaagagaagactattgttgatgtcgtagaaactacaccgaagaaaactattgagagttttggacaaaggacgtctatataccgcggtgttacaaggcatcggtggacaggtagatacgaggcacatttatgggacaatagttgcaaaagagaaggccagactcgcaaaggaagacaagtttatctgggaggttatgacaaagaagaaaaagcagctagggcttacgatttagccgcactaaagtattggggaaccaccactactactaacttccccttgagtgaatatgagaaagaggtagaagagatgaagcacatgacgaggcaagagtatgttgcctctctgcgcaggaaaagtagtggtttctctcgtggtgcatcgatttatcgaggagtaacaaggcatcaccaacatggaaggtggcaagctaggatcggaagagtcgccggtaacaaagacctctacttgggaactttcggcacacaggaagaggctgctgaggcttatgacattgcagccattaaattcagaggattaagcgcagtgactaacttcgacatgaacagatacaatgttaaagcaatcctcgagagcccgagtctacctattggtagttctgcgaaacgtctcaaggacgttaataatccggttccagctatgatgattagtaataacgtttcagagagtgcaaataatgttagcggttggcaaaacactgcgtttcagcatcatcagggaatggatttgagcttattgcagcaacagcaggagaggtacgttggttattacaatggaggaaacttgtctaccgagagtactagggtttgtttcaaacaagaggaggaacaacaacacttcttgagaaactcgccgagtcacatgactaatgttgatcatcatagctcgacctctgatgattctgttaccgtttgtggaaatgttgttagttatggtggttatcaaggattcgcaatccctgttggaacatcggttaattacgatccctttactgctgctgagattgcttacaacgcaagaaatcattattactatgctcagcatcagcaacaacagcagattcagcagtcgccgggaggagattttccggtggcgatttcgaataaccatagctctaacatgtactttcacggggaaggtggtggagaaggggctccaacgttttcagtttggaacgacacttag

SEQ ID NO: 14 - protein, 558 aa, Arabidopsis thaliana
MKNNNNKSSSSSSYDSSLSPSSSSSSHQNWLSFSLSNNNNNFNSSSNPNLTSSTSDHHHPHPSHLSLFQAFSTSPVERQDGSPGVSPSDATAVLSVYPGGPKLENFLGGGASTTTTRPMQQVQSLGGVVFSSDLQPPLHPPSAAEIYDSELKSIAASFLGNYSGGHSSEVSSVHKQQPNPLAVSEASPTPKKNVESFGQRTSIYRGVTRHRWTGRYEAHLWDNSCRREGQSRKGRQVYLGGYDKEDKAARAYDLAALKYWGPTTTTNFPISNYESELEEMKHMTRQEFVASLRRKSSGFSRGASMYRGVTRHHQHGRWQARIGRVAGNKDLYLGTFSTQEEAAEAYDIAAIKFRGLNAVTNFDISRYDVKSIASCNLPVGGLMPKPSPATAAADKTVDLSPSDSPSLTTPSLTFNVATPVNDHGGTFYHTGIPIKPDPADHYWSNIFGFQANPKAEMRPLANFGSDLHNPSPGYAIMPVMQEGENNFGGSFVGSDGYNNHSAASNPVSAIPLSSTTTMSNGNEGYGGNINWINNNISSSYQTAKSNLSVLHTPVFGLE

SEQ ID NO: 15 - DNA, 1677 bp, Arabidopsis thaliana
  source 1..1677: mol_type = other DNA
atgaagaacaataacaacaaatcttcttcttcttctagctatgattcttctttgtctccttcttcttcatcctcctcccaccagaactggctctctttctctctctccaacaataacaacaacttcaattcttcctcaaaccctaatctcacttcctccacatcagatcatcatcatcctcacccttctcacctctctctctttcaagctttctccacttctccagtcgaacggcaagatgggtcaccgggagtttcacccagcgatgccacggcggttctttccgtataccccggcggtcctaaacttgagaacttcctcggcggaggagcctcaacgacgacaacaagaccaatgcaacaagtgcaatctcttggcggcgttgtcttctcttccgacctacagccaccgcttcatcctccgtccgccgccgagatctacgactctgagctcaagtcaatagccgctagcttcctaggaaactactccggtggacactcgtcggaggtctctagcgtacataaacaacaaccgaatcctctagctgtctcagaggcttcgcctactccgaagaagaacgtagagagttttggacaacgtacctcgatttatagaggagtcacaagacatagatggactggaagatacgaagctcatctatgggataatagttgccgaagagaaggccaaagcagaaaaggaagacaagtttatttaggtggttatgataaggaagataaagcagctagagcttacgaccttgcagctcttaagtattggggtcctacaactacgactaatttcccgatatcaaattacgaatctgaacttgaagaaatgaaacacatgactcgacaagagttcgttgcttctttaagacggaaaagcagtggattctctaggggtgcctccatgtacagaggcgtcactagacatcatcagcatggtcgatggcaggcacgaattggaagagttgcaggcaacaaagacctttatcttggcacatttagcactcaagaggaagctgcagaagcttatgatatagcagcgatcaaattccgcggtctaaatgcagtcaccaatttcgacatcagtcgatatgatgtcaaatcaattgctagctgtaatctccctgtgggtggactaatgcctaaaccttctccagcaaccgcagcggctgacaaaaccgttgatctttctccatccgactctccatctctaaccacaccgtccctcacgttcaatgtggcaacaccggtcaatgaccatggaggaactttttaccacactggtataccaatcaaaccagacccggctgatcattattggtccaacatctttggattccaggcaaacccgaaagcagaaatgcgaccattagcaaactttgggtcggatcttcataacccttctcctggttatgctataatgccggtaatgcaggaaggtgaaaacaactttggtggtagttttgttgggtctgatgggtataacaatcattccgctgcatcgaacccggtctcagcaattccgctgtcctcgacaactacaatgagtaacggtaacgaagggtatggtggaaacataaactggattaataacaacatttcaagttcttaccaaactgcaaaatcaaatctctctgttttgcacacaccggtttttgggttggaatga

SEQ ID NO: 16 - protein, 498 aa, Arabidopsis thaliana
MAPPMTNCLTFSLSPMEMLKSTDQSHFSSSYDDSSTPYLIDNFYAFKEEAEIEAAAASMADSTTLSTFFDHSQTQIPKLEDFLGDSFVRYSDNQTETQDSSSLTPFYDPRHRTVAEGVTGFFSDHHQPDFKTINSGPEIFDDSTTSNIGGTHLSSHVVESSTTAKLGFNGDCTTTGGVLSLGVNNTSDQPLSCNNGERGGNSNKKKTVSKKETSDDSKKKIVETLGQRTSIYRGVTRHRWTGRYEAHLWDNSCRREGQARKGRQVYLGGYDKEDRAARAYDLAALKYWGSTATTNFPVSSYSKELEEMNHMTKQEFIASLRRKSSGFSRGASIYRGVTRHHQQGRWQARIGRVAGNKDLYLGTFATEEEAAEAYDIAAIKFRGINAVTNFEMNRYDIEAVMNSSLPVGGAAAKRHKLKLALESPSSSSSDHNLQQQQLLPSSSPSDQNPNSIPCGIPFEPSVLYYHQNFFQHYPLVSDSTIQAPMNQAEFFLWPNQSY

SEQ ID NO: 17 - DNA, 1497 bp, Arabidopsis thaliana
  source 1..1497: mol_type = other DNA
atggctcctccaatgacgaattgcttaacgttttctctgtcaccaatggagatgttgaaatcaactgatcagtctcacttctcttcttcttacgacgattcttctactccttatctcatcgataacttctatgctttcaaagaagaagctgagatagaagctgctgctgcttcaatggcggattcaacaaccttatctacttttttcgatcattctcagactcagattccaaagctggaagatttcctcggtgattcctttgtccgttactctgataaccaaacagagacccaagactcttcttctctcactccattctacgatccacgtcaccgcaccgttgccgaaggagttacagggttcttctctgatcatcatcagccagatttcaagacgataaactcgggaccagaaatcttcgatgactcaacaacttccaacatcggtggtactcatctctccagtcacgtggtggagtcatcaacgacggcgaagttagggtttaacggtgattgcaccaccaccggaggagttttgtctctaggggttaacaacacatcagatcaacctttgagctgtaacaatggcgagagaggtggaaacagtaacaagaagaaaacagtttctaagaaggaaacatcagatgattcaaagaagaagattgtcgaaacattgggacaaagaacttcaatttatcgtggagtcacccgacatagatggactggaagatacgaagcgcatctatgggataacagctgtaggagggaaggtcaagccagaaaaggacgtcaagtgtacttaggtggatatgacaaggaagatagagcagctagagcctatgacttggcagctttaaaatactggggttctactgctactacaaattttccggtctcgagttattcaaaagaacttgaggaaatgaatcacatgaccaagcaagagtttattgcatctcttaggaggaaaagtagcggtttttcgagaggagcttcaatatatagaggtgtcacaaggcatcatcaacaaggtcgctggcaagcaagaatcggccgtgtcgcaggaaacaaagatctttacctcggaacctttgcaaccgaagaggaagcagcagaggcttatgacattgcagccataaagttcagaggaatcaacgcagtaactaactttgagatgaacaggtatgacattgaagctgtcatgaatagttctttacctgtaggaggagcagctgcgaaacgccacaaactcaaactcgctcttgaatctccttcttcatcatcctctgaccataacctccaacaacaacagttgcttccgtcctcttctccctcggatcaaaaccctaactcaatcccatgtggcattccatttgagccttcagttctctattaccaccagaacttctttcagcattatcctttggtctctgactctacaattcaagctcctatgaaccaagctgagtttttcttgtggcctaaccagtcttactaa

SEQ ID NO: 18 - protein, 505 aa, Zea mays
MKLLSSIEQACDICRLKKLKCSKKKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSTAPPTDVSLGDELHLDGEDVAMAHADALDDFDLDMLGDGDSPGPGFTPHDSAPYGALDMADFEFEQMFTDALGIDEYGGDPIQQATAGVSQDTSENPNKTIVPAALPQLTPTLVSLLEVIEPEVLYAGYDSSVPDSAWRIMTTLNMLGGRQVIAAVKWAKAIPGFRNLHLDDQMTLLQYSWMFLMAFALGWRSYRQSSGNLLCFAPDLIINEQRMSLPCMYDQCKHMLFVSSELQRLQVSYEEYLCMKTLLLLSSVPKEGLKSQELFDEIRMTYIKELGKAIVKREGNSSQNWQRFYQLTKLLDSMHEVVENLLTYCFQTFLDKTMSIEFPEMLAEIITNQIPKYSNGNIKKLLFHQK

SEQ ID NO: 19 - DNA, 2130 bp, Zea mays
  source 1..2130: mol_type = other DNA
atggccactgtgaacaactggctcgctttctccctctccccgcaggagctgccgccctcccagacgacggactccacgctcatctcggccgccaccgccgaccatgtctccggcgatgtctgcttcaacatcccccaagattggagcatgaggggatcagagctttcggcgctcgtcgcggagccgaagctggaggacttcctcggcggcatctccttctccgagcagcatcacaagtccaactgcaacttgatacccagcactagcagcacagtttgctacgcgagctcagctgctagcaccggctaccatcaccagctgtaccagcccaccagctccgcgctccacttcgcggactccgtcatggtggcctcctcggccggtgtccacgacggcggttccatgctcagcgcggccgccgctaacggtgtcgctggcgctgccagtgccaacggcggcggcatcgggctgtccatgatcaagaactggctgcggagccaaccggcgcccatgcagccgagggcggcggcggctgagggcgcgcaggggctctctttgtccatgaacatggcggggacgacccaaggcgctgctggcatgccacttctcgctggagagcgcgcacgggcgcccgagagtgtatcgacgtcagcacagggtggtgccgtcgtcgtcacggcgccgaaggaggatagcggtggcagcggtgttgccggtgctctagtagccgtgagcacggacacgggtggcagcggcggcgcgtcggctgacaacacggcaaggaagacggtggacacgttcgggcagcgcacgtcgatttaccgtggcgtgacaaggcatagatggactgggagatatgaggcacatctttgggataacagttgcagaagggaaggacaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgtctcaaggaggccgaggccgcagcgtccgcgcagcaccaccacgccggcgtggtgagctacgacgtcggccgcatcgcctcgcagctcggcgacggcggagccctagcggcggcgtacggcgcgcactaccacggcgccgcctggccgaccatcgcgttccagccgggcgccgccaccacaggcctgtaccacccgtacgcgcagcagccaatgcgcggcggcgggtggtgcaagcaggagcaggaccacgcggtgatcgcggccgcgcacagcctgcaggacctccaccacttgaacctgggcgcggccggcgcgcacgactttttctcggcagggcagcaggccgccgccgcagctgcgatgcacggcctggctagcatcgacagtgcgtcgctcgagcacagcaccggctccaactccgtcgtctacaacggcggggtcggcgatagcaacggcgccagcgccgttggcagcggcggtggctacatgatgccgatgagcgctgccggagcaaccactacatcggcaatggtgagccacgagcagatgcatgcacgggcctacgacgaagccaagcaggctgctcagatggggtacgagagctacctggtgaacgcggagaacaatggtggcggaaggatgtctgcatgggggaccgtcgtctctgcagccgcggcggcagcagcaagcagcaacgacaacattgccgccgacgtcggccatggcggcgcgcagctcttcagtgtctggaacgacacttaa

SEQ ID NO: 20 - protein, 679 aa, Zea mays
MASANNWLGFSLSGQDNPQPNQDSSPAAGIDISGASDFYGLPTQQGSDGHLGVPGLRDDHASYGIMEAYNRVPQETQDWNMRGLDYNGGGSELSMLVGSSGGGGGNGKRAVEDSEPKLEDFLGGNSFVSDQDQSGGYLFSGVPIASSANSNSGSNTMELSMIKTWLRNNQVAQPQPPAPHQPQPEEMSTDASGSSFGCSDSMGRNSMVAAGGSSQSLALSMSTGSHLPMVVPSGAASGAASESTSSENKRASGAMDSPGSAVEAVPRKSIDTFGQRTSIYRGVTRHRWTGRYEAHLWDNSCRREGQSRKGRQVYLGGYDKEDKAARAYDLAALKYWGTTTTTNFPISNYEKELEEMKHMTRQEYIAYLRRNSSGFSRGASKYRGVTRHHQHGRWQARIGRVAGNKDLYLGTFSTEEEAAEAYDIAAIKFRGLNAVTNFDMSRYDVKSILESSTLPVGGAARRLKDAVDHVEAGATIWRADMDGAVISQLAEAGMGGYASYGHHGWPTIAFQQPSPLSVHYPYGQPSRGWCKPEQDAAAAAAHSLQDLQQLHLGSAAHNFFQASSSSTVYNGGAGASGGYQGLGGGSSFLMPSSTVVAAADQGHSSTANQGSTCSYGDDHQEGKLIGYDAAMVATAAGGDPYAAARNGYQFSQGSGSTVSIARANGYANNWSSPFNNGMG

SEQ ID NO: 21 - DNA, 2040 bp, Zea mays
  source 1..2040: mol_type = other DNA
atggcttcagcgaacaactggctgggcttctcgctctcgggccaggataacccgcagcctaaccaggacagctcgcctgccgccggtatcgacatctccggcgccagcgacttctatggcctgcccacgcagcagggctccgacgggcatctcggcgtgccgggcctgcgggacgatcacgcttcttatggtatcatggaggcctacaacagggttcctcaagaaacccaagattggaacatgaggggcttggactacaacggcggtggctcggagctctcgatgcttgtggggtccagcggcggcggcgggggcaacggcaagagggccgtggaagacagcgagcccaagctcgaagatttcctcggcggcaactcgttcgtctccgatcaagatcagtccggcggttacctgttctctggagtcccgatagccagcagcgccaatagcaacagcgggagcaacaccatggagctctccatgatcaagacctggctacggaacaaccaggtggcccagccccagccgccagctccacatcagccgcagcctgaggaaatgagcaccgacgccagcggcagcagctttggatgctcggattcgatgggaaggaacagcatggtggcggctggtgggagctcgcagagcctggcgctctcgatgagcacgggctcgcacctgcccatggttgtgcccagcggcgccgccagcggagcggcctcggagagcacatcgtcggagaacaagcgagcgagcggtgccatggattcgcccggcagcgcggtagaagccgtaccgaggaagtccatcgacacgttcgggcaaaggacctctatatatcgaggtgtaacaaggcatagatggacagggcggtatgaggctcatctatgggataatagttgtagaagggaagggcagagtcgcaagggtaggcaagtttaccttggtggctatgacaaggaggacaaggcagcaagggcttacgatttggcagctctcaagtattggggcactacgacaacaacaaatttccctataagcaactacgaaaaagagctagaagaaatgaaacatatgactagacaggagtacattgcatacctaagaagaaatagcagtggattttctcgtggggcatcaaagtatcgtggagtaactagacatcatcagcatgggagatggcaagcaaggatagggagagttgcaggaaacaaggatctttacttgggcacattcagcaccgaggaggaggcggcggaggcctacgacatcgccgcgatcaagttccgcggtctcaacgccgtcaccaacttcgacatgagccgctacgacgtgaagagcatcctcgagagcagcacactgcctgtcggcggtgcggccaggcgcctcaaggacgccgtggaccacgtggaggccggcgccaccatctggcgcgccgacatggacggcgccgtgatctcccagctggccgaagccgggatgggcggctacgcctcgtacggccaccacggctggccgaccatcgcgttccagcagccgtcgccgctctccgtccactacccgtacggccagccgtcccgcgggtggtgcaaacccgagcaggacgcggccgccgccgcggcgcacagcctgcaggacctccagcagctgcacctcggcagcgcggcccacaacttcttccaggcgtcgtcgagctccacagtctacaacggcggcgccggcgccagtggtgggtaccagggcctcggtggtggcagctctttcctcatgccgtcgagcactgtcgtggcggcggccgaccaggggcacagcagcacggccaaccaggggagcacgtgcagctacggggacgaccaccaggaggggaagctcatcggttacgacgccgccatggtggcgaccgcagctggtggagacccgtacgctgcggcgaggaacgggtaccagttctcgcagggctcgggatccacggtgagcatcgcgagggcgaacgggtacgctaacaactggagctctcctttcaacaacggcatggggtga

SEQ ID NO: 22 - protein, 334 aa, Arabidopsis thaliana
MAAAMNLYTCSRSFQDSGGELMDALVPFIKSVSDSPSSSSAASASAFLHPSAFSLPPLPGYYPDSTFLTQPFSYGSDLQQTGSLIGLNNLSSSQIHQIQSQIHHPLPPTHHNNNNSFSNLLSPKPLLMKQSGVAGSCFAYGSGVPSKPTKLYRGVRQRHWGKWVAEIRLPRNRTRLWLGTFDTAEEAALAYDKAAYKLRGDFARLNFPNLRHNGSHIGGDFGEYKPLHSSVDAKLEAICKSMAETQKQDKSTKSSKKREKKVSSPDLSEKVKAEENSVSIGGSPPVTEFEESTAGSSPLSDLTFADPEEPPQWNETFSLEKYPSYEIDWDSILA

SEQ ID NO: 23 - DNA, 1005 bp, Arabidopsis thaliana
  source 1..1005: mol_type = other DNA
atggcagctgctatgaatttgtacacttgtagcagatcgtttcaagactctggtggtgaactcatggacgcgcttgtaccttttatcaaaagcgtttccgattctccttcttcttcttctgcagcgtctgcgtctgcgtttcttcacccctctgcgttttctctccctcctctccccggttattacccggattcaacgttcttgacccaaccgttttcatacgggtcggatcttcaacaaaccgggtcattaatcggactcaacaacctctcttcttctcagatccaccagatccagtctcagatccatcatcctcttcctccgacgcatcacaacaacaacaactctttctcgaatcttctcagcccaaagccgttactgatgaagcaatctggagtcgctggatcttgtttcgcttacggttcaggtgttccttcgaagccgacgaagctttacagaggtgtgaggcaacgtcactggggaaaatgggtggctgagatccgtttgccgagaaatcggactcgtctctggcttgggacttttgacacggcggaggaagctgcgttggcctatgataaggcggcgtacaagctgcgcggcgatttcgcccggcttaacttccctaacctacgtcataacggatctcacatcggaggcgatttcggtgaatataaacctcttcactcctcagtcgacgctaagcttgaagctatttgtaaaagcatggcggagactcagaaacaggacaaatcgacgaaatcatcgaagaaacgtgagaagaaggtttcgtcgccagatctatcggagaaagtgaaggcggaggagaattcggtttcgatcggtggatctccaccggtgacggagtttgaagagtccaccgctggatcttcgccgttgtcggacttgacgttcgctgacccggaggagccgccgcagtggaacgagacgttctcgttggagaagtatccgtcgtacgagatcgattgggattcgattctagcttag

SEQ ID NO: 24 - DNA, 1519 bp, synthetic construct
  source 1..1519: mol_type = other DNA
aatgaagctcctgtcctccatcgagcaggcctgcgacatctgccgcctcaagaagctcaagtgctccaagaagaagccgaagtgcgccaagtgtctgaagaacaactgggagtgtcgctactctcccaaaaccaagcgctccccgctgacccgcgcccacctcaccgaagtggagtcccgcctggagcgcctggagcagctcttcctcctgatcttccctcgagaggacctcgacatgatcctgaaaatggactccctccaggacatcaaagccctgctcaccggcctcttcgtccaggacaacgtgaacaaagacgccgtcaccgaccgcctggcctccgtggagactgacatgcccctcaccctgcgccagcaccgcatcagcgcgacctcctcctcggaggagagcagcaacaagggccagcgccagttgaccgtctcgacggcccccccgaccgacgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtggggatccaattcagcaagccactgcaggagtctcacaagacacttcggaaaatcctaacaaaacaatagttcctgctgcattaccacagctcacccctaccttggtgtcactgctggaggtgattgaacccgaggtgttgtatgcaggatatgatagctctgttccagattcagcatggagaattatgaccacactcaacatgttaggtgggcgtcaagtgattgcagcagtgaaatgggcaaaggcgataccaggcttcagaaacttacacctggatgaccaaatgaccctgctacagtactcatggatgtttctcatggcatttgccctgggttggagatcatacagacaatcaagtggaaacctgctctgctttgctcctgatctgattattaatgagcagagaatgtctctaccctgcatgtatgaccaatgtaaacacatgctgtttgtctcctctgaattacaaagattgcaggtatcctatgaagagtatctctgtatgaaaaccttactgcttctctcctcagttcctaaggaaggtctgaagagccaagagttatttgatgagattcgaatgacttatatcaaagagctaggaaaagccatcgtcaaaagggaagggaactccagtcagaactggcaacggttttaccaactgacaaagcttctggactccatgcatgaggtggttgagaatctccttacctactgcttccagacatttttggataagaccatgagtattgaattcccagagatgttagctgaaatcatcactaatcagataccaaaatattcaaatggaaatatcaaaaagcttctgtttcatcaaaaatga

SEQ ID NO: 25 - DNA, 1453 bp, synthetic construct
  source 1..1453: mol_type = other DNA
aatgaaagcgttaacggccaggcaacaagaggtgtttgatctcatccgtgatcacatcagccagacaggtatgccgccgacgcgtgcggaaatcgcgcagcgtttggggttccgttccccaaacgcggctgaagaacatctgaaggcgctggcacgcaaaggcgttattgaaattgtttccggcgcatcacgcgggattcgtctgttgcaggaagaggaagaagggttgccgctggtaggtcgtgtggctgccggtgaaccgtcgagcgcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtggggatccgtctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggactcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggagatgctggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagtctga

SEQ ID NO: 26 - DNA, 2226 bp, synthetic construct
  source 1..2226: mol_type = other DNA
atggctagtgaagctcgaaaaacaaagaaaaaaatcaaagggattcagcaagccactgcaggagtctcacaagacacttcggaaaatcctaacaaaacaatagttcctgcagcattaccacagctcacccctaccttggtgtcactgctggaggtgattgaacccgaggtgttgtatgcaggatatgatagctctgttccagattcagcatggagaattatgaccacactcaacatgttaggtgggcgtcaagtgattgcagcagtgaaatgggcaaaggcgataccaggcttcagaaacttacacctggatgaccaaatgaccctgctacagtactcatggatgtttctcatggcatttgccctgggttggagatcatacagacaatcaagtggaaacctgctctgctttgctcctgatctgattattaatgagcagagaatgtctctaccctgcatgtatgaccaatgtaaacacatgctgtttgtctcctctgaattacaaagattgcaggtatcctatgaagagtatctctgtatgaaaaccttactgcttctctcctcagttcctaaggaaggtctgaagagccaagagttatttgatgagattcgaatgacttatatcaaagagctaggaaaagccatcgtcaaaagggaagggaactccagtcagaactggcaacggttttaccaactgacaaagcttctggactccatgcatgaggtggttgagaatctccttacctactgcttccagacatttttggataagaccatgagtattgaattcccagagatgttagctgaaatcatcactaatcagataccaaaatattcaaatggaaatatcaaaaagcttctgtttcatcaaaaatctactagcaaaccggtaacgttatacgacgtcgctgaatacgccggcgtttctcatcaaaccgtttctagagtggttaaccaggcttcacatgttagcgctaaaacccgggaaaaagttgaagctgccatggctgagctcaactacatcccgaaccgtgttgcgcagcagctggctggtaaacaaagcttgctgatcggtgtcgcgacctcgagcttggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccataaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaacacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccactagtggatcggaattcgccaattttaatcaaagtgggaatattgctgatagctcattgtccttcactttcactaacagtagcaacggtccgaacctcataacaactcaaacaaattctcaagcgctttcacaaccaattgcctcctctaacgttcatgataacttcatgaataatgaaatcacggctagtaaaattgatgatggtaataattcaaaaccactgtcacctggttggacggaccaaactgcgtataacgcgtttggaatcactacagggatgtttaataccactacaatggatgatgtatataactatctattcgatgatgaagataccccaccaaacccaaaaaaagagtaa

SEQ ID NO: 27 - DNA, 2464 bp, synthetic construct
  source 1..2464: mol_type = other DNA
aatggcagatacgcgccgacgccagaatcatagctgcgatccctgtcgcaagggcaagcgacgctgtgatgccccggaaaatagaaacgaggccaatgaaaacggctgggtttcgtgttcaaattgcaagcgttggaacaaggattgtaccttcaattggctctcatcccaacgctccaaggcaaaaggggctgcacctagagcgagaacaaagaaagccaggaccgcaacaaccaccagtgaaccatcaacttcagctgcaacaatccctacaccggaaagtgacaatcacgatgcgcctccagtcataaactctcacgacgcgctcccgagctggactcaggggctactctcccaccccggcgaccttttcgatttcagccactctgctattcccgcaaatgcagaagatgcggccaacgtgcagtcagacgcaccttttccgtgggatctagccatccccggtgatttcagcatgggccaacagctcgagaaacctctcagtccgctcagttttcaagcagtccttcttccgccccatagcccgaacacggatgacctcattcgcgagctggaagagcagactacagatccggactcggttaccgatactaatagtgtacaacaggtcgctcaagatggatcgctatggtctgatcggcagtcgccgctactgcctgagaacagtctgtgcatggcctcagacagcacagcacggcgatatgcccgttccacaatgacgaagaatctgatgcgaatctaccacgatagtatggagaatgcactgtcctgctggctgacagagcacaattgtccatactccgaccagatcagctacctgccgcccaagcagcgggcggaatggggcccgaactggtcaaacaggatgtgcatccgggtgtgccggctagatcgcgtatctacctcattacgcgggcgcgccctgagtgcggaagaggacaaagccgcagcccgagccctgcatctggcgatcgtagcttttgcgtcgcaatggacgcagcatgcgcagaggggggctgggctaaatgttcctgcagacatagccgccgatgagaggtccatccggaggaacgcctggaatgaagcacgccatgccttgcagcacacgacagggattccatcattccgggttatatttgcgaatatcatcttttctctcacgcagagtgtgctggatgatgatgagcagcacggtatgggtgcacgtctagacaagctactcgaaaatgacggtgcgcccgtgttcctggaaaccgcgaaccgtcagctttatacattccgacataagtttgcacgaatgcaacgccgcggtaaggctttcaacaggctcccgggaggatctgtcgcatcgacattcgccggtattttcgagacaccgacgccgtcgtctgaaagcccacagcttgacccggttgtggccagtgaggagcatcgcagtacattaagccttatgttctggctagggatcatgttcgatacactaagcgctgcaatgtaccagcgaccactcgtggtgtcagatgaggatagccagatatcatcggcatctccaccaaggcgcggcgctgaaacgccgatcaacctagactgctgggagcccccgagacaggtcccgagcaatcaagaaaagagcgacgtatggggcgacctcttcctccgcacctcggactctctcccagatcacgaatcccacacacaaatctctcagccagcggctcgatggccctgcacctacgaacaggccgccgccgctctctcctctgcaacgcccgtcaaagtcctcctctaccgccgcgtcacgcagctccaaaccctcctctatcgcggcgccagccctgcccgccttgaagcggccatccagagaacgctctacgtttataatcactggacagcgaagtaccaaccatttatgcaggactgcgttgctaaccacgagctcctcccttcgcgcatccagtcttggtacgtcattctagacggtcactggcatctagccgcgatgttgctagcggacgttttggagagcatcgaccgcgattcgtactctgatatcaaccacatcgaccttgtaacaaagctaaggctcgataatgcactagcagttagtgcccttgcgcgctcttcactccgaggccaggagctggacccgggcaaagcatctccgatgtatcgccatttccatgattctctgaccgaggtggcattcctggtagaaccgtggaccgtcgttcttattcactcgtttgccaaagctgcgtatatcttgctggactgtttagatctggacggccaaggaaatgcactagcggggtacctgcagctgcggcaaaattgcaactactgcattcgggcgctgcaatttctgggcaggaagtcggatatggcggcgctggttgcgaaggatttagagagaggtttgaatgggaaagttgacagctttttg

SEQ ID NO: 28 - DNA, 1416 bp, unidentified
  source 1..1416: mol_type = other DNA
gaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaagtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtcaagactggcgaacagttcatacagagtcttttacgactcaatgacaagaagaaaatcttcgtcaacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgacatctccactgacgtaagggatgacgcacaatcccactatccttcgcaagacccttcctctatataaggaagttcatttcatttggagaggacacgctcgagtataagagctcatttttacaacaattaccaacaacaacaaacaacaaacaacattacaattacatttacaatt

SEQ ID NO: 29 - DNA, 204 bp, synthetic construct
  source 1..204: mol_type = other DNA
ctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagat

SEQ ID NO: 30 - protein, 531 aa, Arabidopsis thaliana
MDTLFRLVSLQQQQQSDSIITNQSSLSRTSTTTTGSPQTAYHYNFPQNDVVEECFNFFMDEEDLSSSSSHHNHHNHNNPNTYYSPFTTPTQYHPATSSTPSSTAAAAALASPYSSSGHHNDPSAFSIPQTPPSFDFSANAKWADSVLLEAARAFSDKDTARAQQILWTLNELSSPYGDTEQKLASYFLQALFNRMTGSGERCYRTMVTAAATEKTCSFESTRKTVLKFQEVSPWATFGHVAANGAILEAVDGEAKIHIVDISSTFCTQWPTLLEALATRSDDTPHLRLTTVVVANKFVNDQTASHRMMKEIGNRMEKFARLMGVPFKFNIIHHVGDLSEFDLNELDVKPDEVLAINCVGAMHGIASRGSPRDAVISSFRRLRPRIVTVVEEEADLVGEEEGGFDDEFLRGFGECLRWFRVCFESWEESFPRTSNERLMLERAAGRAIVDLVACEPSDSTERRETARKWSRRMRNSGFGAVGYSDEVADDVRALLRRYKEGVWSMVQCPDAAGIFLCWRDQPVVWASAWRPT

SEQ ID NO: 31 - DNA, 1596 bp, Arabidopsis thaliana
  source 1..1596: mol_type = other DNA
atggatactctctttagactagtcagtctccaacaacaacaacaatccgatagtatcattacaaatcaatcttcgttaagcagaacttccaccaccactactggctctccacaaactgcttatcactacaactttccacaaaacgacgtcgtcgaagaatgcttcaactttttcatggatgaagaagacctttcctcttcttcttctcaccacaaccatcacaaccacaacaatcctaatacttactactctcctttcactactcccacccaataccatcccgccacatcatcaaccccttcctccaccgccgcagccgcagctttagcctcgccttactcctcctccggccaccataatgacccttccgcgttctccatacctcaaactcctccgtccttcgacttctcagccaatgccaagtgggcagactcggtccttcttgaagcggcacgtgccttctccgacaaagacactgcacgtgcgcaacaaatcctatggacgctcaacgagctctcttctccgtacggagacaccgagcaaaaactggcttcttacttcctccaagctctcttcaaccgcatgaccggttcaggcgaacgatgctaccgaaccatggtaacagctgcagccacagagaagacttgctccttcgagtcaacgcgaaaaactgtactaaagttccaagaagttagcccctgggccacgtttggacacgtggcggcaaacggagcaatcttggaagcagtagacggagaggcaaagatccacatcgttgacataagctccacgttttgcactcaatggccgactcttctagaagctttagccacaagatcagacgacacgcctcacctaaggctaaccacagttgtcgtggccaacaagtttgtcaacgatcaaacggcgtcgcatcggatgatgaaagagatcggaaaccgaatggagaaattcgctaggcttatgggagttcctttcaaatttaacattattcatcacgttggagatttatctgagtttgatctcaacgaactcgacgttaaaccagacgaagtcttggccattaactgcgtaggcgcgatgcatgggatcgcttcacgtggaagccctagagacgctgtgatatcgagtttccgacggttaagaccgaggattgtgacggtcgtagaagaagaagctgatcttgtcggagaagaagaaggtggctttgatgatgagttcttgagagggtttggagaatgtttacgatggtttagggtttgcttcgagtcatgggaagagagttttccaaggacgagcaacgagaggttgatgctagagcgtgcagcgggacgtgcgatcgttgatcttgtggcttgtgagccgtcggattccacggagaggcgagagacagcgaggaagtggtcgaggaggatgaggaatagtgggtttggagcggtggggtatagtgatgaggtggcggatgatgtcagagctttgttgaggagatataaagaaggtgtttggtcgatggtacagtgtcctgatgccgccggaatattcctttgttggagagatcagccggtggtttgggctagtgcgtggcggccaacgtaa

SEQ ID NO: 32 - protein, 653 aa, Arabidopsis thaliana
MAESGDFNGGQPPPHSPLRTTSSGSSSSNNRGPPPPPPPPLVMVRKRLASEMSSNPDYNNSSRPPRRVSHLLDSNYNTVTPQQPPSLTAAATVSSQPNPPLSVCGFSGLPVFPSDRGGRNVMMSVQPMDQDSSSSSASPTVWVDAIIRDLIHSSTSVSIPQLIQNVRDIIFPCNPNLGALLEYRLRSLMLLDPSSSSDPSPQTFEPLYQISNNPSPPQQQQQHQQQQQQHKPPPPPIQQQERENSSTDAPPQPETVTATVPAVQTNTAEALRERKEEIKRQKQDEEGLHLLTLLLQCAEAVSADNLEEANKLLLEISQLSTPYGTSAQRVAAYFSEAMSARLLNSCLGIYAALPSRWMPQTHSLKMVSAFQVFNGISPLVKFSHFTANQAIQEAFEKEDSVHIIDLDIMQGLQWPGLFHILASRPGGPPHVRLTGLGTSMEALQATGKRLSDFADKLGLPFEFCPLAEKVGNLDTERLNVRKREAVAVHWLQHSLYDVTGSDAHTLWLLQRLAPKVVTVVEQDLSHAGSFLGRFVEAIHYYSALFDSLGASYGEESEERHVVEQQLLSKEIRNVLAVGGPSRSGEVKFESWREKMQQCGFKGISLAGNAATQATLLLGMFPSDGYTLVDDNGTLKLGWKDLSLLTASAWTPRS

SEQ ID NO: 33 - DNA, 1962 bp, Arabidopsis thaliana
  source 1..1962: mol_type = other DNA
atggcggaatccggcgatttcaacggtggtcaacctcctcctcatagtcctctgagaacaacttcttccggtagtagcagcagcaacaaccgtggtcctcctcctcctcctcctcctcctttagtgatggtgagaaaaagattagcttccgagatgtcttctaaccctgactacaacaactcctctcgtcctcctcgccgtgtctctcaccttcttgactccaactacaatactgtcacaccacaacaaccaccgtctcttacggcggcggctactgtatcttctcaaccaaacccaccactctctgtttgtggcttctctggtcttcccgtttttccttcagaccgtggtggtcggaatgttatgatgtccgtacaaccaatggatcaagactcttcatcttcttctgcttcacctactgtatgggttgacgccattatcagagaccttatccattcctcaacttcagtctctattcctcaacttatccaaaacgttagagacattatcttcccttgtaacccaaatctcggtgctcttcttgaatacaggctccgatctctcatgctccttgatccttcctcttcctctgacccttctcctcaaactttcgaacctctctatcagatctccaacaatccttctcctccacaacagcaacagcagcaccaacaacaacaacaacagcataagcctcctcctcctccgattcagcagcaagaaagagaaaattcttctaccgatgcaccaccgcaaccagagacagtgacggccactgttcccgccgtccaaacaaatacggcggaggctttaagagagaggaaggaagagattaagaggcagaagcaagacgaagaaggattacaccttctcacattgctgctacagtgtgctgaagctgtctctgctgataatctcgaagaagcaaacaagcttcttcttgagatctctcagttatcaactccttacgggacctcagcgcagagagtagctgcttacttctcggaagctatgtcagcgagattactcaactcgtgtctcggaatttacgcggctttgccttcacggtggatgcctcaaacgcatagcttgaaaatggtctctgcgtttcaggtctttaatgggataagccctttagtgaaattctcacactttacagcgaatcaggcgattcaagaagcatttgagaaagaagacagtgtacacatcattgacttggacatcatgcagggacttcaatggcctggtttattccacattcttgcttctagacctggaggacctccacacgtgcgactcacgggacttggtacttccatggaagctcttcaggctacagggaaacgtctttcggatttcgcagataagcttggcctgccttttgagttctgccctttagctgagaaagttggaaacttggacactgagagactcaatgtgaggaaaagggaagctgtggctgttcactggcttcaacattctctttatgatgtcactggctctgatgcacacactctctggttactccaaagattagctcctaaagttgtgacagtagtggaacaagatttgagccacgctggttctttcttaggaagatttgtagaagcaatacattactactctgcactctttgactcactgggagcaagctacggcgaagagagtgaagagagacatgtcgtggaacagcagctattatcgaaagagatacggaatgtattagcggttggaggaccatcgagaagcggtgaagtgaagtttgagagctggagggagaaaatgcaacaatgtgggtttaaaggtatatctttagctggaaatgcagctacacaagcgactctactgttgggaatgtttccttcggatggttacactttggttgatgataatggtacacttaagcttggatggaaagatctttcgttactcactgcttcagcttggacgcctcgttcttag

SEQ ID NO: 34 - DNA, 121 bp, synthetic construct
  source 1..121: mol_type = other DNA
ccgactcattcatccaaataccgagtcgccaaaattcaaactagactcgttaaatgaatgaatgatgcggtagacaaattggatcattgattctctttgatacagatgctataactgagga

SEQ ID NO: 35 - DNA, 21 bp, synthetic construct
  source 1..21: mol_type = other DNA
tacagatgctataactgagga